(19) United States
(12) Patent Application Publication
(10) Pub. No.: US 2018/0296074 A1
(43) Pub. Date: Oct. 18, 2018

(54) IMPLANTABLE OPTICAL PROBES AND SYSTEMS AND METHODS FOR IMPLANTATION OF OPTICAL PROBES

(71) Applicant: INSCOPIX, INC., Palo Alto, CA (US)

(72) Inventors: Mark O. TRULSON, San Jose, CA (US); Sam J. MALANOWSKI, San Francisco, CA (US)

(21) Appl. No.: 15/955,887

(22) Filed: Apr. 18, 2018

Related U.S. Application Data
(63) Continuation of application No. PCT/US2016/058307, filed on Oct. 21, 2016.
(60) Provisional application No. 62/244,660, filed on Oct. 21, 2015.

(51) Int. Cl. A61B 1/00; G02B 21/36; G02B 23/24; G02B 27/00

(52) U.S. Cl. CPC A61B 1/00149; A61B 1/00045; A61B 1/00179; H04N 5/2256; G02B 21/361; G02B 23/2446; G02B 27/0025; A61B 1/00096

(57) ABSTRACT

Provided herein are optical probes to enhance the accessible volume imaged with a microscope, and systems and methods for inserting the optical probes into an object for imaging of the interior of the object. The object can be a tissue of a living organism. The probe can continuously image the space in the vicinity of the probe as the probe is inserted into the object. The probe can image a sample at an angle great than 0° relative to the implantation axis of the probe. The probe can be connected to a surface of the object by a cuff. The cuff can comprise one or more surface features to increase a surface area of the cuff that attaches to the surface of the object. The cuff can be held by a clamp while the probe is inserted into the object for imaging.

### CROSS-REFERENCE

This application claims the benefit of U.S. Provisional Application No. 62/244,660, filed on Oct. 21, 2015, which disclosure is hereby incorporated by reference in its entirety.

### BACKGROUND

Optical probes can be implanted in an object to extend the depth a microscope can address to explore and image features below the surface of the object. Probes typically comprise a relay lens that transmits light from a light source to a sample below the surface of the object in which the probe is implanted. Similarly, the probe transmits light from the sample to the microscope image sensor to generate an image of the sample. In some cases, an organ (e.g., brain) can be imaged using a microscope probe. In some instances, probes may be ideally suited for imaging deep organ (e.g. brain) tissue and/or cells. The optical probe may enable imaging of one or more sections of the organ that are below normally accessible regions of the organ.

### SUMMARY

A need exists for probes of different lengths, of different diameters, or for probes that can alter the viewing angle of the probe. These probes can decrease or can enlarge the field of view of the microscope. The probes can increase the depth of features in the object accessible to imaging with the microscope. Optical elements can be optically and/or mechanically coupled to, or integrated with the probes to correct optical aberrations (e.g. chromatic, spherical aberrations).

A need exists to provide a controlled and efficient method of inserting an optical probe into an object for imaging. Provided herein is an apparatus that can control implantation of an optical probe for imaging of internal features and structures inside of an object. The internal features and structures may not be visible from the outside of the object. The internal features may not be visible from an ambient environment surrounding the object. In some methods the probe can collect images of the inside of the object looking for a sample of interest by trial and error. In the systems and methods described herein, the probe can continuously image the inside of the object as the probe is inserted into the object such that trial and error methods for finding the sample of interest can be avoided.

An object can be imaged repeatedly over hours, days, week, months, and/or years. Systems and methods described herein permit stable and repeatable insertion of an optical probe for repeated imaging of a sample of interest contained within an object. A collar can be provided permanently or removably attached to an object. The collar can direct and stabilize the probe during insertion and imaging. In some cases, the collar can be permanently or removably attached to the object with an adhesive. The systems and methods described herein provide an attachment surface area for the adhesive. The systems and methods described herein reduce the likelihood of adhesive spreading to other parts of the microscope, probe, and/or a probe stabilization device.

Thus, in one aspect, a method of implanting an optical probe into an object for imaging one or more interior features of the object is presented. The method comprises: supporting an optical probe with a stabilization device, wherein the optical probe is in optical communication with a microscope; inserting the optical probe into the object; continuously imaging one or more interior features of the object while inserting the probe into the object; and displaying the one or more interior features of the object while using the microscope inserting the probe into the object.

In some embodiments, the method further comprises stopping inserting the optical probe into the object at a location when a sample of interest is detected. In some embodiments, the method further comprises removing the clamp from the cuff while maintaining the location of the probe. In some embodiments, the stabilization device is connected to a stereotaxic manipulator rod. In some embodiments, the stereotaxic manipulator rod is configured to allow movement of the optical probe with respect to at least three axes. In some embodiments, the stereotaxic manipulator rod is configured to allow translation or rotation of the optical probe. In some embodiments, the stabilization device comprises a clamp connected to a cuff, wherein the cuff is supporting the optical probe. In some embodiments, the method further comprises analyzing, with aid of one or more processors, images of the one or more interior features while inserting the probe into the object. In some embodiments, a field of view of the optical probe is increased while inserting the probe into the object. In some embodiments, a field of view of the optical probe is decreased while inserting the probe into the object. In some embodiments, the one or more interior features of the object are displayed in real time.

In another aspect, a device configured to implant an optical probe into an object for imaging is provided. The device comprises: a cuff that supports an optical probe that is in optical communication with a microscope, the cuff (1) comprising a surface that connects to an outer surface of the object with an adhesive and (2) configured to prevent adhesive from leaking out of a contact area between the surface of the cuff that connects and the outer surface of the object; and a clamp that removably connects to the cuff and is connected to a stereotaxic manipulator rod configured to control the device when the optical probe is inserted into the object.

In some embodiments, one or more images are collected as the optical probe is inserted into the object. In some embodiments, the device is configured to locate a feature of interest while the optical probe is being inserted into the object. In some embodiments, the stereotaxic manipulator rod is configured to allow movement of the optical probe with respect to at least three axes. In some embodiments, the stereotaxic manipulator rod is configured to allow translation or rotation of the optical probe. In some embodiments, the probe comprises a relay lens. In some embodiments, the relay lens is a gradient index lens. In some embodiments, the GRIN lens has an angled surface. In some instances, the angle is defined with respect to the optical axis of the GRIN lens. In some embodiments, the angle is between 30 degrees and 60 degrees. In some embodiments, the angle is a 45 degree angle. In some embodiments, the angled surface is produced by grinding the GRIN lens at 45 degrees. In some embodiments, the angled surface is produced by etching the GRIN lens. In some embodiments, the etching is accomplished by chemical etching means. In some embodiments, the etching is accomplished by physical etching means. The 45 degree angle may produce fewer sharp edges, resulting in a lessened amount of damage during insertion of a probe into tissue. This may yield a probe which is better for use in primates and other animals. In some embodiments, the relay lens is located at a distal end of the probe. In some embodiments, the device further comprises a corrective optical element. In some embodiments, the corrective optical element comprises a refractive or diffractive optical element. In some embodiments, the corrective optical element is mechanically aligned with the relay lens by a housing or sheathing. In some embodiments, the device further comprises an optical element configured to alter a viewing angle of the probe. In some embodiments, the optical element is a prism.

A probe may have a proximal end closer to a microscope and a distal end further from the microscope. The distal end of the probe may include an angled surface. The angled surface may have any angular value, such as those described elsewhere herein. The angled surface may be an angled end of a GRIN lens, or may be an angled surface of a prism attached to a distal end of the GRIN lens. The angled surface may cover an entirety of a distal end of the probe. The angled surface may have a circular or elliptical shape. The cross-sectional area of the angled surface in a plane perpendicular to a longitudinal axis of the GRINS lens may match the cross-sectional area of the GRIN lens in a plane perpendicular to the longitudinal axis of the GRIN lens. Rotating an optical probe about its longitudinal axis may cause a field of view to change. The angled surface may cause the field of view to be substantially to a side of the probe. For example, if the angled surface has a 45 degree angle, the field of view may be substantially perpendicular to a longitudinal axis of the probe. Rotating the optical probe may cause the field of view to circle around the probe. Adjusting a position of the optical probe along a longitudinal axis may cause the field of view to move along the longitudinal axis as well in a corresponding manner.

In another aspect, a method of extending an accessible volume in an object for imaging with optical probes coupled to a microscope is provided. The method comprises: imaging at a plurality of different depths in the object, wherein the plurality of different depths are set by a length of the optical probe; and imaging at a plurality of different fields of view, wherein the plurality of different fields of view are set by a diameter of the optical probes, wherein the imaging at a plurality of different depths and imaging at a plurality of different fields of view are undertaken in a single imaging session.

In some embodiments, imaging at a plurality of different depths or imaging at a plurality of different fields of view is accomplished co-linear with the microscope's optical axis. In some embodiments, imaging at a plurality of different depths or imaging at a plurality of different fields of view is accomplished non-collinear with the microscope's optical axis by addition of an optical element to the optical probes. In some embodiments, the optical element is a prism. In some embodiments, the method further comprises correcting monochromatic optical aberrations by addition of refractive or diffractive optical elements to the optical probe. In some embodiments, the method further comprises correcting poly-chromatic optical aberrations by addition of refractive or diffractive optical elements to the optical probe.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a schematic view of a device configured to provide controlled implantation of an optical probe into an object.

FIG. 2 shows a detailed view of a device configured to provide controlled implantation of an optical probe into an object.

FIG. 3 shows a cross section view of a probe fitted in a cuff in a device configured to provide controlled implantation of an optical probe into an object.

FIG. 4 shows an overhead view or a device configured to provide controlled implantation of an optical probe into an object.

FIG. 5 shows an optical probe configured to collect images along an implantation axis of the probe.

FIG. 6 shows a microscope that can be provided in optical communication with an implantable probe.

FIG. 7 shows a process of using a probe stabilization device to implant a probe to image a sample contained in an object.

FIG. 8 shows a process of using a stabilization device to insert a probe into an object.

FIG. 9 shows a microscope attached to a stabilization device.

FIG. 10 shows a detailed view of a cuff of a stabilization device.

FIG. 11 illustrates an endoscopic “prism probe” that comprises a cylindrical angled prism and a GRIN lens that fits inside an implantable glass cannula to provide adjustable viewing depth and viewing direction.

FIG. 12 illustrates the refraction of light rays at the interface between the cannula wall and tissue that can give rise to image blur and distortion.

FIG. 13 shows a comparison of examples of image distortion observed for an uncorrected 1 mm square prism probe to that observed for an uncorrected 1 mm cylindrical prism probe.

FIG. 14 shows a comparison of examples of image distortion observed for images captured using cylindrical prism probes having corrective optical elements attached.

FIG. 15 shows a plot of an unconstrained toroidal object field surface sag as a function of field position.

FIG. 16 shows a plot of corrective optical element surface sag as a function of position for the corrective optical element used to achieve the unconstrained toroidal object field shown in FIG. 15.

FIG. 17 shows a plot of object field surface sag as a function of field position when the radii of curvature of the toroidal object field are constrained. The prism probe axis is indicated.

FIG. 18 shows an example of image distortion for an image captured using a 1 mm cylindrical prism probe comprising a cylindrical corrective optical element designed to achieve a constrained toroidal object field.

FIG. 19 shows a plot of surface sag as a function of position for a cylindrical corrective optical element.

FIG. 20 shows ray traces in a view of one embodiment of a cylindrical prism probe optimized for a constrained toroidal object field.

FIG. 21 illustrates one embodiment of a cylindrical prism probe set to the maximum depth within an implantable cannula.

FIG. 22 illustrates one embodiment of a cylindrical prism probe set to the minimum depth within an implantable cannula.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

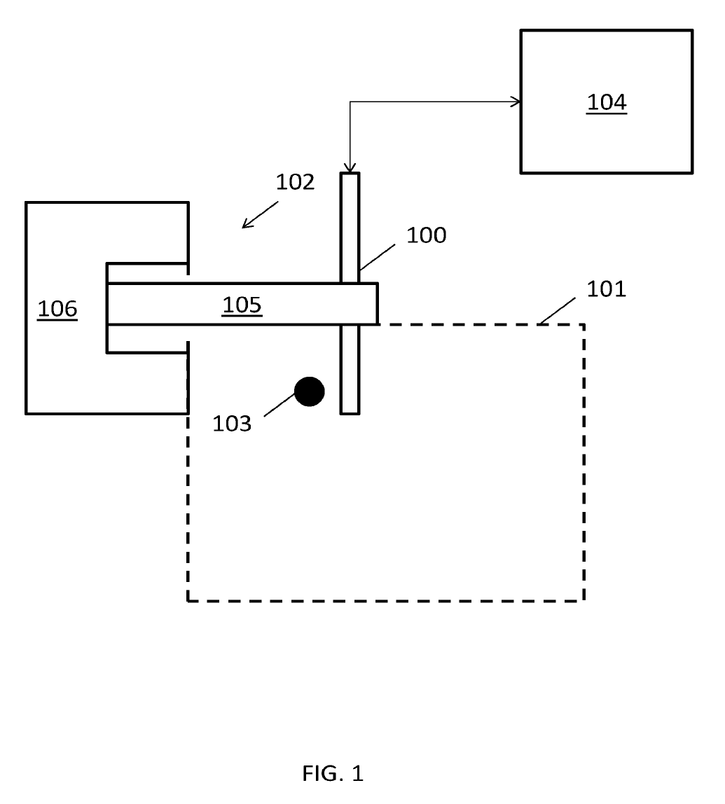

Measuring of one or more internal samples contained in an object can be performed by inserting a probe into the object and aligning an imaging plane of the probe with the internal sample of interest. FIG. 1 shows an optical probe 100 being inserted into an object 101 by a stabilization device 102. The stabilization device can provide support for the optical probe while the probe is inserted into the object. The stabilization device can control a rate of insertion of the probe into the object. The stabilization device can prevent lateral movement of the probe during insertion. The stabilization device can prevent lateral movement of the probe during imaging with the probe. The stabilization device can prevent rotation of the probe during insertion. The stabilization device can prevent rotation of the probe during imaging with the probe. The stabilization device can prevent vibration of the probe during insertion and/or imaging.

In some cases, inserting the probe may comprise inserting only a portion of the probe. In some cases, inserting the probe may comprise inserting the entirety of the probe. Inserting the probe may comprise inserting any portion of the probe. Inserting the probe may comprise inserting up to 10%, 20%, 30%, 40%, 50%, 60%, 70%, 80%, 90%, or 100% of the probe. Inserting the probe may comprise inserting at least 10%, 20%, 30%, 40%, 50%, 60%, 70%, 80%, 90%, or 100% of the probe. Inserting the probe may comprise inserting a fraction of the probe within a range defined by any two of the preceding values. Any description herein of inserting a percentage or fraction of a probe may apply to the percentage or fraction of the volume of the probe, the length of the probe, the width of the probe, the mass of the probe, or any other dimension of the probe.

The stabilization device can comprise a cuff 105 and a clamp 106. The cuff can be adhered, at least temporarily, to a surface. The surface can be a surface of the object. In some cases, the cuff can be adhered to a surface that coats, covers, contacts, or resides next to the object. In some cases, the cuff can be adhered to tissue or bone that is next to the object. The cuff can be adhered to skin or bone that coats, covers, contacts, or resides next to the object. The cuff can be adhered to the surface temporarily or permanently. The cuff can be removed from the surface with warm water. The cuff can be removed from the surface using a solvent. The cuff can be adhered to the surface for a duration of time sufficient to permit imaging of the object. The cuff can be adhered to the surface for one or more minutes, hours, days, weeks, months, or years. The cuff can be adhered to the surface with an adhesive. The adhesive can be a water soluble adhesive. The adhesive can be glue, paste, epoxy, cement (e.g., dental cement), double sided tape, medical tape, or any other suitable adhesive. The cuff can be adhered to the surface with one or more hardware components, for example, screws. The cuff can be adhered to the surface with one or more straps. The clamp can be removably attached to the cuff. The cuff can be fitted in the clamp. A perimeter of the cuff can be held in a vice of the clamp. The clamp can be oriented above and/or to a side of the object. The cuff can be fitted in the clamp during insertion of the probe. The cuff can be fitted in the clamp while the cuff is being adhered to the surface.

The probe 100 can be an optical element. The probe can transmit light. The probe can transmit light from a light source to a region of the object for imaging of a sample of interest. Light that is emitted and/or reflected off of the sample can be transmitted through the probe to a microscope system in optical communication with the probe. The probe can spectrally filter light such that only light of a predetermined range of wavelengths is transmitted through the probe. The probe can be permanently attached to the cuff. The probe can be integrated with the cuff. The probe can be built into the cuff. The probe and the cuff can form one integral part.

The object can contain a sample of interest 103 to be imaged by the optical probe. The sample of interest can be a structure or feature that is internal to the object. The sample of interest can be a structure or feature that cannot be observed from the surface of the object. The sample of interest can be a structure or feature that requires insertion of the optical probe into the object for observation of the sample of interest. The sample of interest can be a structure that is not visible from an outside surface of the object. In some cases, at least a portion of the probe can directly contact the sample during imaging of the sample. An image of the sample of interest can be optically transmitted to a microscope 104 that is optically coupled to the probe. In some cases, the probe can continuously transmit one or more images to the microscope while the probe is being inserted into the object.

In some cases, the precise location of a sample of interest (e.g., sample) inside of the object may not be known. In some systems a trial and error method can be used to determine the location of the sample of interested inside of the object. In the trial and error approach a probe can be inserted into the object a given distance and one or more images can be collected in the vicinity of the probe at the inserted distance. The image can be analyzed to determine if the image at least partially contains the sample of interest. If the sample of interest is not at least partially contained in the image the probe can be inserted at a different distance and another image can be taken and analyzed. This process can be repeated until the sample of interest is located. The trial and error method can be time consuming. The trial and error method can require unnecessary repeated insertion of the probe into the object which can cause damage to the object.

Provided herein are systems and methods for simultaneously inserting a probe into an object and imaging the interior features of the object such that a location of a sample of interest with an unknown location inside of the object can be detected while inserting the probe into the object. In some cases, one or more images may be collected with a microscope in optical communication with the probe while inserting the probe into the object. In some cases, one or more interior features of the object may be imaged while inserting the probe into the object. In some cases, the interior features of the object may be displayed while inserting the probe into the object. The interior features may be displayed on a display terminal. The display terminal may be an external device. The display terminal may be an electronic screen, monitor, smartphone, tablet, or any other display device as known to one having skill in the art. The interior features may be displayed on a display terminal in real time.

Detecting the object while inserting the probe can eliminate the need for the trial and error method and therefore decrease the amount on time needed to locate the sample. Detecting the object while inserting the probe can minimize damage to the object. Detecting while inserting the probe can allow the stopping of the insertion of the probe when a sample of interest is encountered. The probe can provide imaging of at least a portion of the object in real time. The probe can provide imaging of at least a portion of the object while the probe is inserted into the object. A user can observe a field of view of the microscope as the probe is inserted into the object in real time. The field of view may comprise the portion of an object that may be observed using the probe at any given point in time. A field of view may be altered as a user is inserting or retracting the probe within the object. The field of view may be altered as a user is rotating the probe about a longitudinal axis within the object. The field of view may be altered as the user is changing an angle or position of a distal end of the probe. A user can stop or adjust insertion of the probe when the sample of interest is observed. The user can increase or decrease the area of the field of view in real time as the probe is inserted into the object (e.g., via software). The user can increase or decrease the area of the field of view in real time after the probe is inserted into the object (e.g., via software).

The object can include living tissue. The object can include non-living tissue. In some cases, the object can comprise a tissue of a living or non-living organism. The tissue can be muscle tissue, cardiac tissue, organ tissue, brain tissue, epithelial tissue, breast tissue, fatty tissue, or any other tissue. The object can comprise one or more organs (e.g., brain, heart, stomach). The object can have a volume equal to or less than about 0.01 cm3, 0.1 cm3, 0.5 cm3, 1 cm3, 5 cm3, 10 cm3, 20 cm3, 30 cm3, 40 cm3, 50 cm3, 60 cm3, 70 cm3, 80 cm3, 90 cm3, 100 cm³, or 1000 cm³. The object can have a volume less than 0.01 cm³. The object can have a volume greater than 1000 cm³. The object can have a volume that falls between any of the values listed herein.

The sample of interest can be a feature or structure inside of the object that is of interest to a user of the probe. In an example where the object comprises tissue, the sample of interest can be a structure that is inherent to the specific tissue type. The sample of interest can be a valve, node, cell mass, membrane or other structure inherent to the tissue type. In an example where the object comprises tissue, the sample of interest can be a structure that is associated with an abnormality. The structure associated with an abnormality can be a tumor, cell mass, cyst, ulcer, polyp, fluid mass, or any other tissue abnormality. In some cases, the probe can insert into brain tissue to observe one or more samples inside of the brain tissue. In some cases, the sample of interest can be below the surface of a brain. The sample of interest can be one or more cells. The sample of interest can be one or more nerve cells (e.g., neurons). The sample of interest can be one or more activated neurons. The sample of interest can be two or more neurons that are interacting. The sample of interest can be one or more neurons that fire in response to a stimulus.

The probe can be implanted into a living organism (e.g., subject) for imaging of one or more samples of interest inside an object (e.g., organ) of the organism. In some cases, at least a portion of the probe can directly contact the sample during imaging of the sample. The probe can be held in place during imaging of the one or more samples of interest by a stabilization device. At least a fraction of the stabilization device may be mounted onto a living organism or a non-living organism. In some instances, the stabilization device may be mounted to an exterior of an organism (e.g., over skin of the organism). The stabilization device may be mounted to a bone structure (e.g., skull) of the organism. The organism can be anesthetized while the stabilization device is mounted to the bone structure and/or while the probe is inserted into the organism. In some cases the organism can be awake up during imaging with the probe.

The microscope may be mounted to a head of the organism and used to image brain tissue of the organism. The microscope may be mounted to the organism and used to image any other tissue on or within the organism. Examples of samples may include any biological sample or tissue, such as nervous tissue (e.g., brain tissue), muscle tissue, connective tissue, or epithelial tissue. An organism may be a human subject or an animal subject. In some embodiments, animal subjects may include rodents (e.g., mice, rats, rabbits, guinea pigs, gerbils, hamsters), simians, canines, felines, avines, insects, or any other types of animals. In some instances, the subjects may weigh less than about 50 kg, 40 kg, 30 kg, 20 kg, 15 kg, 10 kg, 5 kg, 3 kg, 2 kg, 1 kg, 750 grams, 500 grams, 400 grams, 300 grams, 200 grams, 100 grams, 75 grams, 50 grams, 40 grams, 30 grams, 25 grams, 20 grams, 15 grams, 10 grams, 5 grams, 3 grams, or 1 gram.

In some embodiments, part or all of the microscope can be inserted into a living organism or a non-living organism. The microscope can be connected to a probe inserted into an organism. The probe may or may not contact a tissue of the organism. The microscope can be used in vivo, or in vitro. In some instances, the microscope may be used in vivo for a subject that is conscious. The microscope may be used in vivo for a subject that is not anesthetized. The microscope may be used in vivo for a subject that may be freely moving or mobile. The subject may be able to freely walk around an environment while the microscope is connected to (e.g., mounted on, inserted within) the subject. The subject may be able to freely walk around an environment while the microscope is imaging a sample of the subject. The subject can be exposed to stimuli while the probe is inserted in the subject. The outward behavior of the subject can be observed while simultaneously imaging an internal organ and/or tissue of the subject. In some cases, the probe can remain implanted in a tissue and/or organ of the live being for minutes, hours, days, weeks, and/or months. The microscope can be removed from and reattached to the implanted probe at various points in time ranging from minutes, hours, days, weeks, and/or months to collect one or more images of a sample of interest.

A small microscope, such as those having dimensions as described elsewhere herein, may be advantageous to provide little interference with activities of the subjects, or to be used with smaller subjects, such as those having characteristics described herein. The probe can be optically coupled to the microscope. The microscope may be used to image a sample on or within the organism. The probe can extend the optical path of the microscope such that the microscope can image samples that would otherwise be outside of a depth of field of an objective lens of the microscope. In some cases, the probe can be inserted into an object such that the microscope in optical communication with the probe can access and image a sample inside of the object. An end of the probe can be inserted into the object a distance of at least about 0.001 mm, 0.005 mm, 0.01 mm, 0.05 mm, 0.1 mm, 0.5 mm, 1 mm, 5 mm, 10 mm, 20 mm, 30 mm 40 mm, 50 mm, 60 mm, 70 mm, 80 mm, 90 mm, or 100 mm from a surface of the object.

The probe can be inserted into an object for imaging of one or more samples of interest inside of the object. The probe can be inserted by a stabilization device. In some cases the probe can be a cylindrical probe with a small diameter. The probe can have a diameter of less than or equal to about 100 mm, 90 mm, 80 mm, 70 mm, 60 mm, 50 mm, 40 mm, 30 mm, 20 mm, 15 mm, 10 mm, 5 mm, 4 mm, 3 mm, 2 mm, 1 mm, 0.5 mm, 0.1 mm, 0.05 mm, 0.01 mm, 0.005 mm, or 0.001 mm. The small diameter of the probe can reduce damage to the organism. In some instances, the small diameter may decrease the stiffness and/or structural rigidity of the probe such that the probe can bend and/or break when a compressive force is applied to the probe. The stabilization device described elsewhere herein can be configured to apply sufficient force to the probe to hold the probe in place without bending or breaking the probe. The stabilization device can be tightened with fine adjustment such that the probe can be secured in the device without breaking or bending. The stabilization device can be configured to apply a uniform pressure around the outer diameter of the probe when the probe is held in place by the stabilization device.

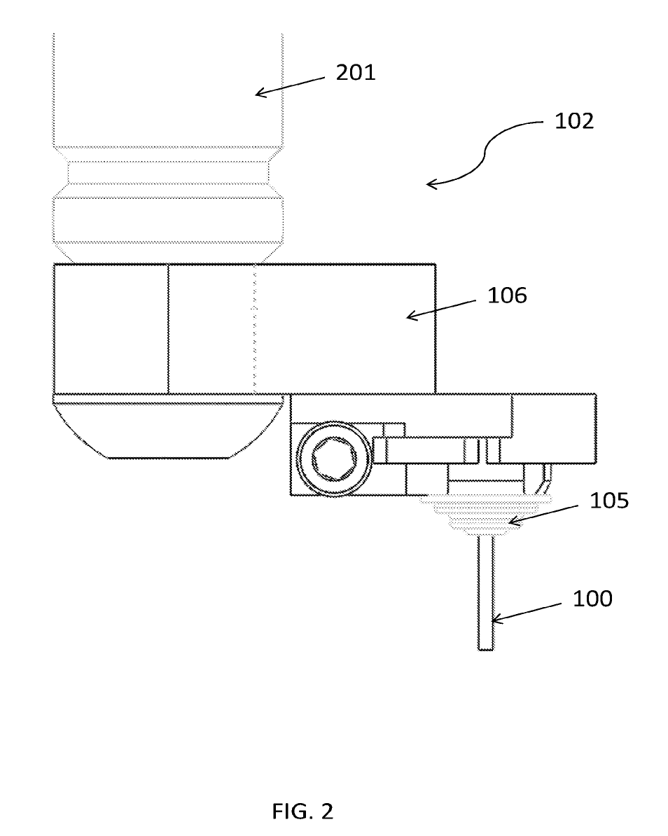

FIG. 2 shows a stabilization device 102 configured to control implantation of a probe for imaging of a sample of interest below a surface. The stabilization device can control insertion, extraction, and incremental movement of the probe in an object. In some cases, the sample of interest can be below the surface of an organ. In some cases, the sample of interest can be below the surface of a brain. The sample of interest can be one or more cells. The sample of interest may not be exposed to an ambient environment. The probe may have to penetrate a surface of the object in order to access, view, and/or contact the sample of interest. The sample of interest can be one or more nerve cells (e.g., neurons). The probe can be implanted into the object while a microscope in optical communication with the probe is imaging a sample in the vicinity of the probe.

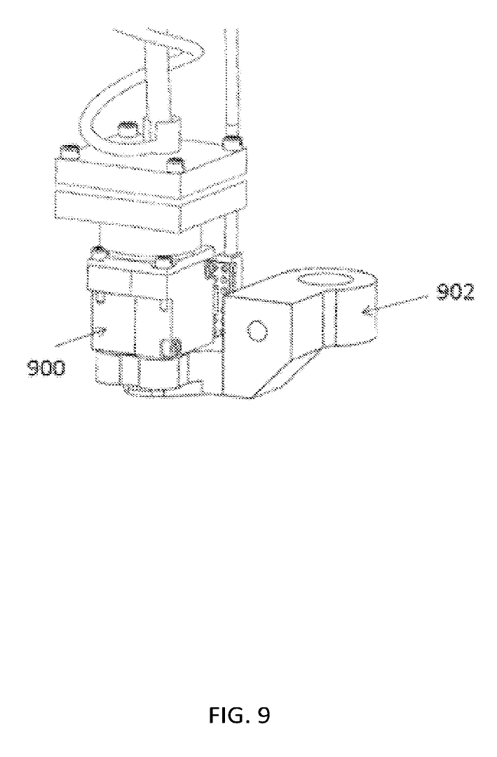

In some instances, a microscope can be attached to the stabilization device. FIG. 9 shows a microscope 900 attached to a stabilization device 902. At least a portion of the microscope can be housed in the stabilization device. The microscope can be in optical communication with the probe 100. The stabilization device can provide a rigid and/or controlled connection between the microscope 900 and the probe such that the microscope can image a sample while the probe is inserted into and/or extracted from an object. The stabilization device can control insertion and/or extraction of the probe at a rate of at least about 0.0001 mm/s, 0.001 mm/s, 0.01 mm/s, 0.1 mm/s, 1 mm/s, 5 mm/s, 10 mm/s, 20 mm/s, 30 mm/s, 40 mm/s, 50 mm/s, 60 mm/s, 70 mm/s, 80 mm/s, 90 mm/s, 100 mm/s, 500 mm/s, 1000 mm/s. The rate of insertion can be less than 0.0001 mm/s. The rate of insertion can be greater than 1000 mm/s. The rate of insertion can fall between any of the values stated herein.

The stabilization device can permit the probe to be inserted into the object without jostling and/or lateral movement of the probe such that an image of a sample in the vicinity of the probe can be detected continuously with high resolution. The resolution can be sufficient to identify one or more structures present in the sample of interest. The resolution can be sufficient to identify cell structures. The resolution can be sufficient to observe nerve cell dendrites and axons. The resolution can be sufficient to observe neuronal activity. In some instances, the resolution is equal to, or less than about 0.01 μm, 0.1 μm, 0.5 μm, 1 μm, 1.5 μm, 2 μm, 2.5 μm, 3 μm, 4 μm, 5 μm, 10 μm, 15 μm, 20 μm, 25 μm, 30 μm, 35 μm, 40 μm, 45 μm, 50 μm, 0.1 mm, 0.2 mm, 0.3 mm, 0.5 mm, 1 mm, 2 mm, 5 mm, 10 mm, or 20 mm. In some instances, the resolution may be in between any of the foregoing values. In some instances, the resolution is within a range of about 0.1 μm to about 1 mm.

In some cases the microscope can be a small microscope. The microscope can have a maximum dimension less than about 5 inches, 4 inches, 3 inches, 2 inches, 1.5 inches, 1 inch, 0.75 inch, or 0.5 inches. A maximum dimension of the microscope as used herein may refer to any dimension of the microscope (e.g., length, width, height, diameter) that is greater than the other dimensions of the microscope. The microscope may have a volume of less than or equal to about 10 cubic inches, 7 cubic inches, 5 cubic inches, 4 cubic inches, 3 cubic inches, 2 cubic inches, 1.5 cubic inches, 1 cubic inch, 0.7 cubic inches, 0.5 cubic inches, 0.3 cubic inches, or 0.1 cubic inch. The microscope may have a lateral cross section (e.g., footprint) or less than or equal to about 5 square inches, 4 square inches, 3 square inches, 2 square inches, 1.5 square inches, 1.2 square inches, 1 square inch, 0.9 square inches, 0.8 square inches, 0.7 square inches, 0.6 square inches, 0.5 square inches, 0.3 square inches, or 0.1 square inches. The microscope may have a weight of less than or equal to about 10 grams, 7 grams, 5 grams, 4 grams, 3.5 grams, 3 grams, 2.5 grams, 2 grams, 1.5 grams, 1 gram, 0.5 grams, or 0.1 grams. The small dimensions may useful for applications where a subject may be small, to provide reduced interference with activities of the subject by the microscope. Furthermore the small dimension of the microscope can permit the stabilization device to similarly have small dimensions such that the stabilization device does not interfere with other optical and/or electrical probes that can be implanted in the vicinity of the optical probe. A small lateral cross-section is useful when the subject is small and/or there is a limited space or area where the microscope may be mounted.

The stabilization device can be attached to a stereotaxic manipulator rod 201, as shown in FIG. 2. The stereotaxic manipulator rod 201 can be configured to move with respect to at least three axes. The stereotaxic manipulator rod 201 can be configured to cause rotation and/or translation of an object attached to the manipulator rod, for example the probe. The stereotaxic manipulator rod 201 can be configured to move in an x-direction, y-direction, and/or z-direction in a reference frame of the object being imaged by the probe. The stereotaxic manipulator rod 201 can be in communication with a computer system (not shown). The computer system can comprise a memory storage device. A map of an area containing a subject to be imaged by the device can be stored on the memory storage device. The map can comprise spatial and or relational data pertaining to a tissue, organ, and or bone structure of an animal.

The stabilization device can comprise a clamp 106 and a cuff 105. The stabilization device can be configured to position a probe 100. The stabilization device can be used during insertion of the probe into the object. At least a portion of the stabilization device can be removed from the probe after the probe is inserted into the object. The stabilization device may or may not be used after the probe is inserted into the object. The probe 100 can be fitted in the cuff. The cuff 105 can be permanently attached to the probe 100. The cuff 105 can be removably attached to the probe 101. In some cases, a plurality of probes with different diameters can be attached to the cuff. The cuff can be adjustable such that the cuff can accommodate probes of different diameters. Probes with different diameters and/or different optical properties can be fitted in the cuff for different imaging applications. Different optical properties can include focal length, magnification, resolution, and/or other optical properties. In some cases, probes of different lengths can be fitted in the cuff for different imaging applications.

The clamp can provide an interface to connect the cuff 105 to the stereotaxic manipulator rod 201. The clamp can be permanently attached to the cuff. The clamp can be removably attached to the cuff. The clamp can be permanently attached to the stereotaxic manipulator rod. The clamp can be removably attached to the stereotaxic manipulator rod. The clamp may comprise a rod interface that may permit the clamp to be attached to the stereotaxic manipulator rod. The rod interface may contact the stereotaxic manipulator rod when the clamp and rod are attached. The rod interface may include one or more mating features or connecting features that may allow the rod to be attached to the clamp. The clamp may comprise a cuff interface that may permit the clamp to be attached to the cuff. The cuff interface may contact the cuff when the clamp and cuff are attached. The cuff interface may include one or more mating features or connecting features that may allow the cuff to be attached to the clamp.

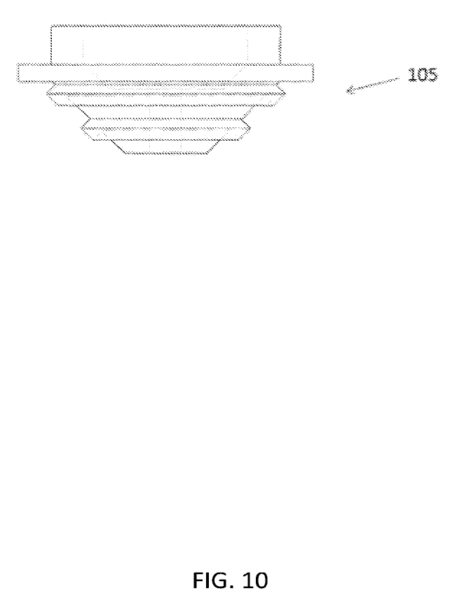

FIG. 10 shows a detailed view of a cuff of a stabilization device. The cuff 105 can be attached to an object that contains a sample to be imaged by the probe. The cuff can support a probe inserted in the object. The cuff can keep a probe inserted in the object. The cuff can hold the probe at an insertion location in the object. The cuff can be attached to the object with an adhesive and/or one or more fasteners. In some cases, the cuff can be attached to the object with dental cement. The cuff 105 can be attached to a structure that contains a sample to be imaged by the probe. The cuff can be attached to a bone that at least partially encloses an organ or tissue to be imaged by the probe. In some cases the cuff can be attached to a skull that contains brain tissue to be imaged by the probe. The cuff can be sized and shaped such that it does not interfere with other optical and/or electrical probes that are implanted in the vicinity of the device. The cuff can be sized and shaped such that cuff comprises a sufficient surface area for attaching to an object (e.g., bone, skull). The cuff can comprise a flange that provides a surface area for insertion of one or more fasteners (e.g., screws) for attachment to an object. In some cases a surface of the cuff that contacts an object can comprise ridges, grooves, a pattern of raised bumps, a pattern of raised lines, or any other features that provide additional surface area for contacting the object. In some cases a surface area of the cuff that contacts an object can be equal to or greater than about 1 mm², 2 mm², 5 mm², 10 mm², 15 mm², 20 mm², 25 mm², 30 mm², 40 mm², 50 mm², 75 mm2, 100 mm², 125 mm², 150 mm², 200 mm², 250 mm², or 300 mm²

The cuff can be attached to an object with a paste-like adhesive. The paste-like adhesive can be spreadable on a surface. The paste-like adhesive can drip on to and/or leak on to other components of the device. The paste-like adhesive can be flowable. The cuff can comprise structures that prevent adhesive from leaking and/or dripping from the contact surface and contacting the clamp, the probe, and/or any other parts of the device. The cuff can comprise a lip that prevents adhesive from leaking, dripping, or oozing out on to the clamp, the probe, and/or any other parts of the device. In some cases, the cuff can comprise a chamfered edge configured to prevent adhesive from leaking, dripping, or oozing out on to the clamp, the probe, and/or any other parts of the device.

The cuff can have a tapered shape. The cuff can have a conical shape. The cuff can be sized and shaped such that the cuff can fit into a hole with a counter-bore. The cuff can be hollow such that one or more optical elements can be housed in the cuff. One or more optical elements can be housed in the concave portion of the cuff. The inner volume of the cuff 105 can be equal to or greater than about 1 mm³, 2 mm³, 5 mm³, 10 mm³, 15 mm³, 20 mm³, 25 mm³, 30 mm³, 40 mm³, 50 mm³, 75 mm³, 100 mm³, 125 mm³, 150 mm³, or 200 mm³. A surface of an implanted lens can be housed in the cuff An objective lens of the microscope can be housed in the cuff. The cuff can be sized and shaped such that the location of the objective lens can be adjusted along the optical axis of the objective lens. Moving the location of the objective lens along the optical axis of the objective lens can provide fine focusing of the microscope. The objective lens can be moved along the optical axis of the objective lens during imaging, insertion of the probe into the object, and/or removal of the probe from the object.

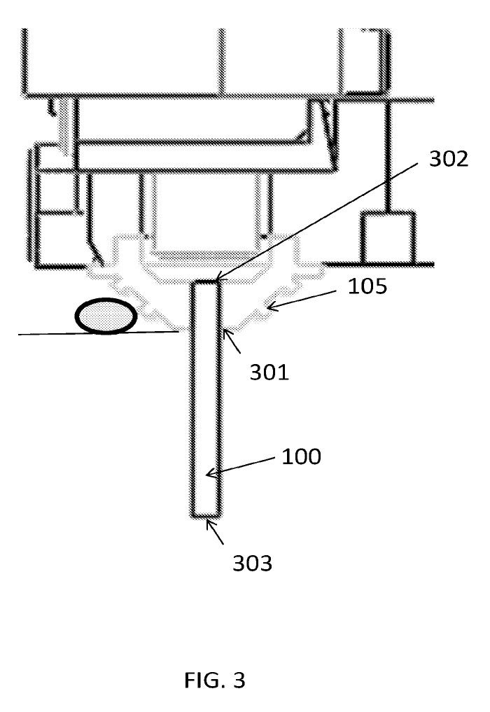

FIG. 3 shows a cross section view of a probe 100 fitted in a cuff 105. At least a fraction of the probe can be inserted into an opening 301 provided in the cuff When the probe is inserted into the opening 301 the probe can be centered within the field of view of a microscope that is in optical communication with the probe. The microscope can be a miniature microscope. The probe can comprise a relay lens. A surface of the relay lens can be on an end 302 of the probe. The surface of the lens can be contained in the cuff. The surface of the lens contained in the cuff can be protected from dirt, dust, and other contaminants. The surface of the lens contained in the cuff can be isolated from adhesive that connects the cuff to an object. In some cases the relay lens can comprise one or more GRIN lenses. In some cases, the one or more GRIN lenses may have a surface 303. In some cases, the surface is an angled surface. In some instances, the angle is defined with respect to the optical axis of the GRIN lens. In some cases, the angle is between 30 degrees and 60 degrees. In some cases, the angle is a 45 degree angle. In alternative embodiments, the angle may be about 15 degrees or less, 30 degrees or less, 45 degrees or less, 60 degrees or less, or 75 degrees or less. Alternatively, the angle may be greater than any of the values described. In some cases, the angled surface may be produced by grinding the one or more GRIN lenses at 45 degrees. In some cases, the angled surface may be produced by etching the one or more GRIN lenses. In some cases, the etching may be accomplished by chemical etching means. In some cases, the etching may be accomplished by physical etching means.

The clamp can hold the cuff in a position such that the cuff is aligned with an optical axis of the microscope. The clamp can maintain alignment of the cuff and an optical axis of the microscope while the probe is inserted into an object for imaging. Imaging can occur continuously while the probe is inserted into the object. A position of the cuff relative to the optical axis of the microscope when the probe is inserted into an object at a first depth can be identical to a position of the cuff relative to the optical axis of the microscope when the probe is inserted into an object at a second depth.

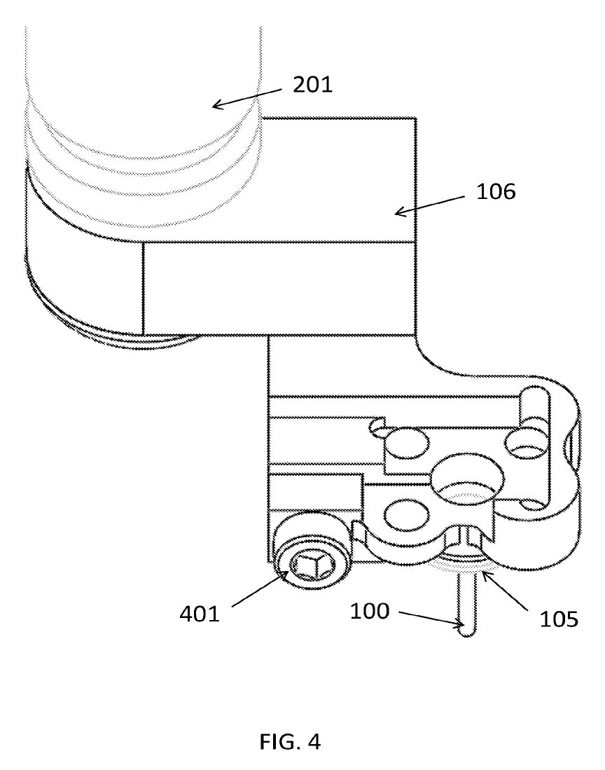

FIG. 4 shows an overhead view of the clamp 106. The clamp 106 can be sized and shaped such that when the clamp is observed from an overhead perspective, such as the perspective shown in FIG. 4, the cuff can be at least partially visible. A user can access the cuff when the cuff is attached to the clamp. A user can adjust the position of the cuff when the cuff is attached to the clamp. A user can apply adhesive to the cuff when the cuff is attached to the clamp. In some cases the cuff and the clamp can be made from materials with different colors (e.g., different colored plastics or different colored metals) such that a user can easily distinguish between the cuff and the clamp. In some cases the cuff and the clamp can be painted with different colors such that a user can easily distinguish between the cuff and the clamp. In some cases one of the cuff and the clamp can have a shiny finish and the other can have a dull finish such that a user can easily distinguish between the cuff and the clamp.

The clamp can comprise one or more tightening screws 401. The clamp can comprise a single tightening screw. The tightening screw can connect the cuff to the clamp. The cuff can be connected to the clamp permanently. The cuff can be removably connected to the clamp. The cuff can be removed from the clamp while maintaining a location of the optical probe. The screw can be a set screw such that tightening the screw pushes against a surface to hold the cuff in place in the clamp. The screw can comprise threads such that the threads can be screwed into a threaded hole provided on either or both of the clamp and the cuff when the screw is tightened. In some instances, the screw threads may contact threads in the clamp such that tightening the screw will deform the clamp and tighten the clamp around an interface with the cuff.

In some cases the probe 100 that is inserted by the device 102 can have a diameter, a length, and be positioned longitudinally with respect to the objective lens, so as to either decrease or enlarge the field of view and/or depth range of the microscope and probe combination. The probe can comprise a gradient index or other relay lenses. The gradient index lens can have a pitch of at least about 1/2, 2/2, 3/2, 4/2, 5/2, 6/2, 7/2, 8/2, 9/2, 10/2, 11/2, 12/2, or 13/2. The gradient index lens can have diameters ranging from 0.001 mm to 5 mm. The GRIN lens may have an angled surface. The angle may be a 45 degree angle. The angled surface may be produced by grinding the one or more GRIN lenses at 45 degrees. The angled surface may be produced by etching the one or more GRIN lenses. The etching may be accomplished by chemical etching means. The etching may be accomplished by physical etching means. An additional corrective optical element (not shown) that corrects optical aberration including field curvature can be optically coupled to or mechanically integrated with the probe. In some embodiments, the corrective optical element may be incorporated into a miniature microscope that is optically coupled with or mechanically integrated with the probe. In some embodiments, the corrective optical element may be incorporated within the probe itself. The corrective optical element can be mechanically aligned with the probe gradient-index (GRIN) lens by a probe housing or sheathing.

The corrective element can comprise one or more refractive and/or a diffractive optical elements. The corrective element may comprise one or more aspheric diffractive optical elements. The corrective optical element (not shown) can be optically coupled to or mechanically integrated with the objective. The corrective optical element and the objective lens can be removably connected. In some cases, the corrective optical element and the objective lens can be connected by sliding the housing comprising the objective lens over the corrective optical element. The corrective optical element can be fitted in the microscope housing with a force fit connection. One or more set screws can be provided to hold the optical element in the housing. The corrective optical element can screw into the housing. The corrective optical element can be fitted in the housing when the corrective optical element and the objective lens are optically coupled. In some cases, the corrective optical element and one or more components that are connected to the corrective optical element (e.g., prism or other optical elements) can remain in an object while the housing comprising the objective lens is removed from the corrective optical element.

The corrective optical element may be designed to correct for wavelengths in the ultraviolet, visible, or near-infrared regions of the electromagnetic spectrum. In some cases, the corrective optical element may be designed to correct for wavelengths in a range from 200 nm to 1300 nm, 200 nm to 1200 nm, 200 nm to 1100 nm, 200 nm to 1000 nm, 200 nm to 900 nm, 200 nm to 800 nm, 200 nm to 700 nm, 300 nm to 700 nm, or 400 nm to 700 nm.

In some cases, the probe that is inserted by the device 102 can be configured to image a sample at an angle that is not aligned with an implantation axis of the probe. The probe can provide a viewing angle that is not aligned (not collinear) with the implantation axis of the probe. The probe can observe a sample of interest that is at an angle relative to a longitudinal axis of the probe. For example, the probe can observe a sample of interest that is located at an angle of 90° relative to the longitudinal axis of the probe. The probe can be configured to provide only one viewing angle. The probe can be configured to provide any viewing angle in the range of 0° to 180°. The viewing angle of the probe can be variable. The probe can provide continuous adjustment of the viewing angle. The probe can provide adjustment of the viewing angle in discrete predetermined increments. In some cases, the viewing angle of the probe can be adjusted without removing the probe from the object in which the probe is implanted.

The probe can comprise a gradient index or other relay lens and an optical element configured to alter the viewing angle of the probe. The gradient index lens can have a pitch of at least about 1/2, 2/2, 3/2, 4/2 5/2, 6/2, 7/2, 8/2 9/2, 10/2, 11/2, 12/2 or 13/2. In some cases, the optical element can comprise an angled surface of the GRIN lens. In some cases, the angle is a 45 degree angle. In some cases, the angled surface may be produced by grinding the GRIN lens at 45 degrees. In some cases, the angled surface may be produced by etching the GRIN lens. In some cases, the etching may be accomplished by chemical etching means. In some cases, the etching may be accomplished by physical etching means. The optical element can comprise a prism. The optical element can comprise a lens. The optical element can comprise a liquid lens. In some embodiments, the probe may comprise a GRIN lens with an angled surface built-in. Alternatively, a separate optical element, such as a prism, with an angled surface may be attached to the GRIN lens or within the same optical path as the GRIN lens. Any description herein of an angled GRIN lens may apply to an arrangement with an additional angled optical element, or vice versa.

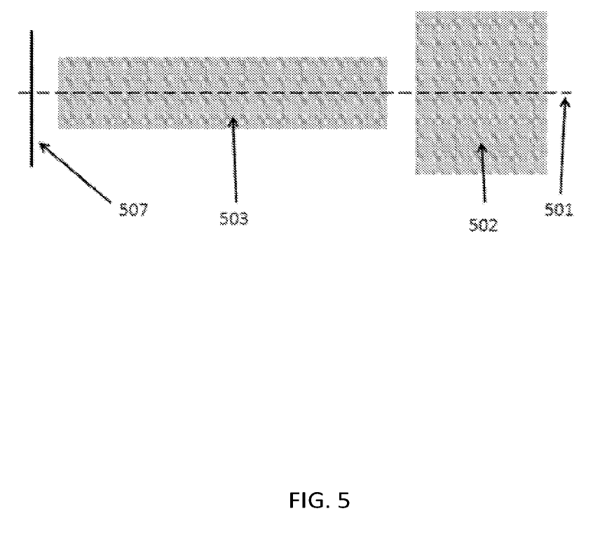

FIG. 5 shows an example of a probe utilizing a relay lens or lens group 503 which can image a sample along the implantation axis 501 of the probe. The object plane 507 of the sample may be substantially perpendicular to the implantation axis of the probe. The sample may be located near a distal end of the probe. In some cases, the distal end of the probe can be separated from the sample by at least about 0.00001 mm, 0.0001 mm, 0.001 mm, 0.01 mm, 0.1 mm, 0.5 mm, 1 mm, 2 mm, 3 mm, 4 mm, 5 mm, 10 mm, 15 mm, 20 mm, 25 mm, 30 mm, 40 mm, 50 mm, 60 mm, 70 mm, 80 mm, 90 mm, or 100 mm. An additional corrective optical element (not shown) that corrects optical aberration including field curvature can be optically coupled to or integrated with the probe 100. The corrective optical element can be mechanically aligned with the probe GRIN lens by a probe housing or sheathing. The corrective element can comprise one or more refractive and/or a diffractive optical elements. The corrective optical element (not shown) can be optically coupled to or mechanically integrated with the microscope objective 502. The corrective optical element and the objective lens can be removably connected. In some cases, the corrective optical element and the objective lens can be connected by sliding the housing comprising the objective lens over the corrective optical element. The corrective optical element can be fitted in the microscope housing with a force fit connection. One or more set screws can be provided to hold the optical element in the housing. The corrective optical element can screw into the housing. The corrective optical element can be fitted in the housing when the corrective optical element and the objective lens are optically coupled. In some cases, the corrective optical element and one or more components that are connected to the corrective optical element can remain in an object while the housing comprising the objective lens is removed from the corrective optical element.

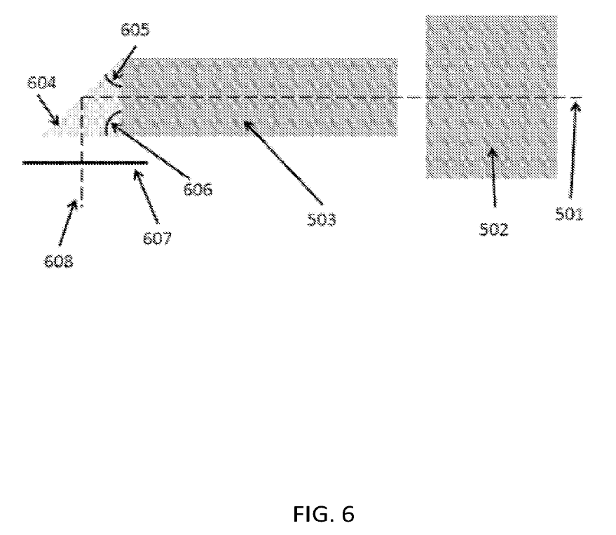

FIG. 6 shows an example of a probe 100 in which a prism 604 coupled to a relay lens 503 can image a sample along a viewing axis 608 that is not aligned with the implantation axis 501 of the probe. In some instances, the object plane 607 of the sample may be substantially along a longitudinal axis of the probe. In some instances, the object plane of the sample may be substantially parallel to an implantation axis 501 of the probe. In some instances, the object plane 607 of the sample may form an angle equal to or greater than about 0°, 5°, 10°, 15°, 20°, 25°, 30°, 35°, 40°, 45°, 50°, 55°, 60°, 70°, 80°, 90°, 100°, 110°, 120°, 130°, 140°, 150° together with the implantation axis of the probe. For instance, a field of view of the probe may be directly in line with the longitudinal axis of the probe. Alternatively, the field of view of the probe may be offset from the longitudinal axis of the probe. For instance, the field of view of the probe may be orthogonal to the longitudinal axis of the probe. The field of view may have any angle relative to the longitudinal axis of the probe, such as any of the angles described herein. An additional corrective optical element or element group (not shown) that corrects optical aberration including field curvature can be optically coupled to or mechanically integrated with the probe 100. The corrective optical element can be mechanically aligned with the probe GRIN lens by a probe housing or sheathing. The corrective element can comprise one or more refractive and/or a diffractive optical elements. The corrective optical element (not shown) can be optically coupled to or mechanically integrated with the objective 502. The corrective optical element and the objective lens can be removably connected. In some cases, the corrective optical element and the objective lens can be connected by sliding the housing comprising the objective lens over the corrective optical element. The corrective optical element can be fitted in the microscope housing with a force fit connection. One or more set screws can be provided to hold the optical element in the housing. The corrective optical element can screw into the housing. The corrective optical element can be fitted in the housing when the corrective optical element and the objective lens are optically coupled. In some cases, the corrective optical element and one or more components that are connected to the corrective optical element (e.g., prism or other optical elements) can remain in an object while the housing comprising the objective lens is removed from the corrective optical element.

An optical element (e.g. a prism) 604 may be configured to alter the viewing angle of the probe. The optical element can be affixed to an end of the GRIN or other relay lens 503. The optical element can be an angled surface of the GRIN lens. In some cases, the angle is a 45 degree angle. In some cases, the angled surface may be produced by grinding the GRIN lens at 45 degrees. In some cases, the angled surface may be produced by etching the GRIN lens. In some cases, the etching may be accomplished by chemical etching means. In some cases, the etching may be accomplished by physical etching means. The optical element 604 configured to alter the viewing angle of the probe can be affixed to an end of the GRIN or relay lens 503 opposite an end of the GRIN or relay lens facing the objective lens 502. The optical element 604 configured to alter the viewing angle of the probe can comprise a prism 604. The prism can comprise one or more reflective surfaces. In some cases the prism can be a triangular prism. The prism can comprise three rectangular faces. Two rectangular faces can be oriented at a right angle 606 to each other. Two of the faces can be oriented at an angle 605 to each other. In some cases, the angle 605 between two of the faces can have any value between 0° and 180°, thereby altering the viewing angle of the probe. Adjustment of angle 606 can help to reduce imaging artifacts introduced by the prism.

The angle can be adjustable. The angle can be varied while the probe is implanted in an object. The angle can be adjusted without moving parts. The angle can be adjusted using a liquid crystal. In some cases, the liquid lens can respond to a change in a voltage applied across the crystal. One or more electrical contact can be provided on the liquid crystal to apply a voltage across the liquid crystal. Alternatively, the viewing angle can be changed by rotating the prism about a joint. The prism can be rotated by an actuator. The prism can be rotated by a piezo electric device.

In some cases, one or more of the sides of the optical element (e.g. prism) can be coated with a reflective material. The reflective material can be metal, metal foil or one or more layers of dielectric material. The side of the prism coated with a reflective material can reflect at least about 50%, 60%, 70%, 80%, 90% or 100% of light incident on the side of the prism coated with the reflective material.

The optical element (prism) can contact a sample in the object plane 607 of the probe. In some cases, the prism can be separated from the sample in the object plane of the probe by at least about 0.00001 mm, 0.0001 mm, 0.001 mm, 0.01 mm, 0.1 mm, 0.5 mm, 1 mm, 2 mm, 3 mm, 4 mm, 5 mm, or 10 mm. A sample in the object plane of the probe can be imaged on to an intermediate plane, optically conjugate with the object plane, between the probe and the microscope objective lens. The sample may be located near a distal end of the probe. In some cases, the distal end of the probe can be separated from the sample by at least about 0.00001 mm, 0.0001 mm, 0.001 mm, 0.01 mm, 0.1 mm, 0.5 mm, 1 mm, 2 mm, 3 mm, 4 mm, 5 mm, 10 mm, 15 mm, 20 mm, 25 mm, 30 mm, 40 mm, 50 mm, 60 mm, 70 mm, 80 mm, 90 mm, or 100 mm.

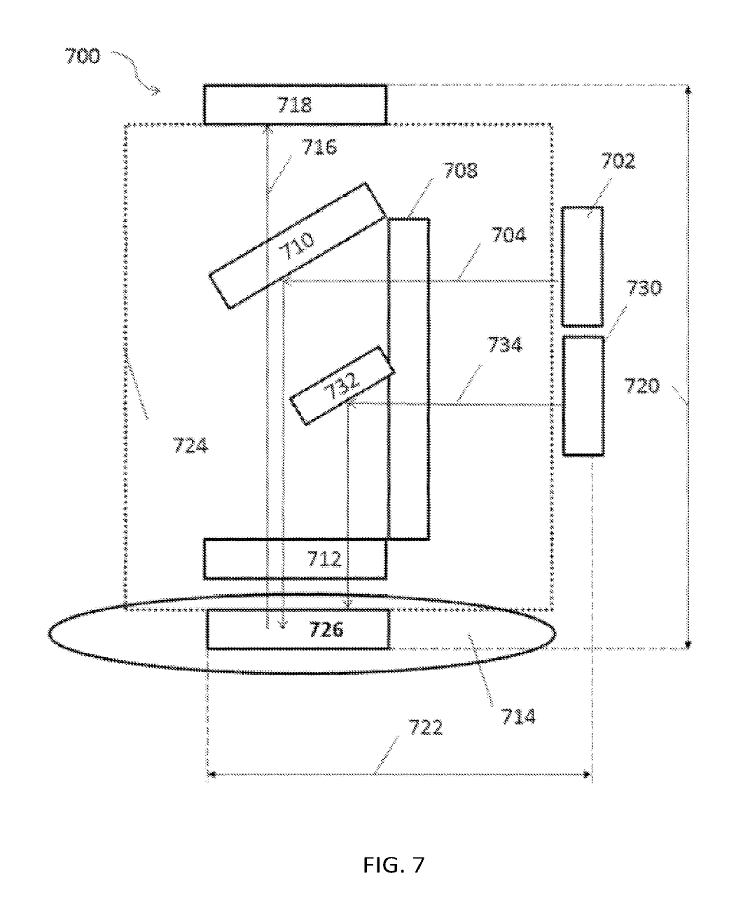

The probe can be in optical communication with a microscope for imaging one or more samples in the object plane of the probe. FIG. 7 shows a block diagram of a microscope 700 that can be in optical communication with the imaging probe. One or more of the components of the microscope 700 can be housed on the stabilization device 102 described elsewhere herein.

The microscope device 700 may include a number of components within the dimensions 720 and 722. Not shown is a further dimension, which extends perpendicular to the dimensions 720 and 722. Although not necessarily limited thereto, each of these dimensions can be less than an inch. In some cases, dimension 720 can be at most about 0.001 inch, 0.01 inch, 0.05 inch, 0.1 inch, 0.2 inch, 0.3 inch, 0.4 inch, 0.5 inch, 0.6 inch, 0.7 inch, 0.8 inch, 0.9 inch, 1 inch, 1.5 inches, 2 inches, or 5 inches. In some cases, dimension 722 can be at most about 0.001 inch, 0.01 inch, 0.05 inch, 0.1 inch, 0.2 inch, 0.3 inch, 0.4 inch, 0.5 inch, 0.6 inch, 0.7 inch, 0.8 inch, 0.9 inch, 1 inch, 1.5 inches, 2 inches or 5 inches. In some cases the dimension extending perpendicular to the dimensions 720 and 722 can be at most about 0.001 inch, 0.01 inch, 0.05 inch, 0.1 inch, 0.2 inch, 0.3 inch, 0.4 inch, 0.5 inch, 0.6 inch, 0.7 inch, 0.8 inch, 0.9 inch, 1 inch, 1.5 inches, 2 inches, or 5 inches.

The microscope 700 can include a light directing arrangement 702. This light directing arrangement 702 can direct imaging light 704 to the sample. The light directing arrangement can include one or more light sources. The one or more light sources can be on board the microscope. The one or more light sources can be off board the microscope. Light emission from the one or more light sources can be transmitted to the light directing arrangement through a light transmission element, for example, a fiber optic element. In a particular implementation, the optical source 702 is a light-emitting-diode (LED) or an organic light-emitting-diode (OLED). In some cases, the optical source 702 can be a laser. The imaging light 704 from the light source 702 can be directed by an optical arrangement 724 to a surface of the probe 714 in optical communication with the microscope 700 for imaging of a sample of interest. In some cases, the optical arrangement 724 can be housed on the stabilization device and the light source can be off board the stabilization device. Light from the light source can be transmitted from the off board light source to the optical arrangement on the stabilization device by an optical transmission device (e.g., fiber optic). In some cases the light source can be housed on the stabilization device.

In some cases, the probe can deliver a first light emission to the sample of interest for imaging (e.g., imaging light) 704 and a second light emission 734 (e.g., stimulation light) from a second light source 730 to the sample of interest to stimulate the sample of interest. The stimulation light can be directed to the sample through a dichroic mirror 732. The probe can deliver the imaging light and the stimulation light to the sample simultaneously. The probe can permit a user to view a sample of interest's response to the stimulation light.

The probe can be in contact with the sample of interest. The probe can deliver light 704 to the sample of interest. The optical arrangement can include one or more of objective lens 712, (dichroic beamsplitter) mirror 710 and excitation filter 708 and an emission filter (not depicted). The probe can collect emitted and/or reflected light 616 from the sample of interest. Light 716 from the probe 714 can be directed from/by the objective lens to an image capture circuit 718. The image capture circuit can comprise one or more photo detectors. The microscope 700 may be configured to direct light from and capture image data for a field of view 726. The field of view 726 of the microscope can be determined by a field of view of the probe. The field of view 726 of the microscope can comprise a field of view of the probe.

In various embodiments of the present disclosure, the microscope 700 can also include one or more of an image-focusing optical element (e.g., an achromatic lens) and an emission filter. These and other elements can help control optical properties of the microscope 700.

Consistent with one embodiment, the depicted elements are each integrated into a relatively small area, e.g., within a single housing having dimensions 720, 722. The stabilization device can comprise the housing. The total volume of the housing can be at most about 5 in³, 3 in³, 1 in³, 0.75 in³, 0.5 in³, 0.25 in³, or 0.1 in³. The housing may be formed from a single part or multiple pieces. The housing may partially or completely enclose one or more of the components described herein. The housing may be optically opaque and may prevent light from outside the microscope from entering the microscope. In some instances, light may only enter the interior of the microscope through the objective lens.

Such integration of the various components can be particularly useful for reducing the length the optical pathway from the optical source 702 to the probe 714 and back to the image capture circuit 718. The reduction of this optical pathway can be part of the configuration parameters that facilitate a number of different properties and capabilities of the microscope 700. For example, in certain embodiments the microscope can provide images with a resolution to 1 um for an imaging field of view with an area of at least about 0.01 mm², 0.05 mm², 0.1 mm², 0.5 mm², 1 mm², 2 mm², 3 mm², 4 mm², or 5 mm²

In some cases, the image capture circuit 718 can comprise an array of optical sensors. An optical arrangement 724 is configured to direct light 704 of less than about 1 mW (various embodiments provide for a higher excitation power, e.g., 100 mW) to a probe 714 with a field of view of that is at least 0.5 mm². In some cases, the field of view can be at least about 0.01 mm², 0.1 mm², 0.2 mm², 0.3 mm², 0.4 mm², 0.5 mm², 0.6 mm², 0.7 mm², 0.8 mm², 0.9 mm², 1 mm², 1.5 mm², 2 mm2, 3 mm2, 4 mm2, 5 mm2, 10 mm², or 50 mm². The field of view can be between any of the values listed. The field of view can be smaller than 0.01 mm². The field of view can be greater than 50 mm²

In some cases, the light source can be configured to direct epi-fluorescence emission caused by incident imaging light to the image capture circuit 718. In various embodiments, the field of view can be at least 1 mm². The light directing arrangement and image capture circuit 718 can each be configured sufficiently close to the probe 714 to provide at least 2.5 μm resolution for an image of the field of view. In other embodiments, the light directing arrangement and image capture circuit 718 can be configured to provide at least 1 μm resolution. Images captured by the probe can provide resolution similar to or identical to the resolution of the microscope without the probe. Images captured by a probe comprising a prism, a GRIN lens, and a corrective optical element may provide resolution that is similar or identical to the resolution of the microscope without the probe. Images captured by a probe comprising a GRIN lens having a 45° angle at one end and a corrective optical element may provide resolution that is similar or identical to the resolution of the microscope without the probe. In some embodiments, images captured by a probe having a 45° angle at one end and no corrective optical element may provide resolution that is similar or identical to the resolution of the microscope without the probe through the use of image processing to remove image distortion. Alternatively images captured with the probe may have an improved resolution compared to images captured without the probe, or images captured without the probe may have an improved resolution compared to images captured with the probe. In some instances, the resolution is equal to, or less than about 0.01 μm, 0.1 μm, 0.5 μm, 1 μm, 1.5 μm, 2 μm, 2.5 μm, 3 μm, 4 μm, 5 μm, 10 μm, 15 μm, 20 μm, 25 μm, 30 μm, 35 μm, 40 μm, 45 μm, 50 μm, 0.1 mm, 0.2 mm, 0.3 mm, 0.5 mm, 1 mm, 2 mm, 5 mm, 10 mm, or 20 mm. In some instances, the resolution may be in between any of the foregoing values. In some instances, the resolution is within a range of about 0.1 μm to about 1 mm. In some embodiments, the aforementioned resolution may be achieved at the center of the field of view. In some embodiments, the aforementioned resolution may be achieved across the field of view. In certain embodiments, the excitation optical power at the specimen is variable and can be in the range of 100 μW-100 mW, depending upon the particular configuration and imaging constraints.

A typical light source can deliver light of up to 37 lumens or 6 mW. The total illumination power delivered to the sample of interest may not exceed about 10 mW, 9 mW, 8 mW, 7 mW, 6 mW, 5 mW, 4 mW, 3 mW, 2 mW, or 1 mW. It is not, however, necessarily a requirement that the light source provide light of such power. Moreover, the amount of light received by the sample of interest is less than (relative to an attenuation factor) the amount of light provided by the light source. For instance, the attenuation of one embodiment results in 6 mW at the light source corresponding to 1 mW excitation power delivered at the target object. Similarly, to deliver 100 mW of excitation power at the specimen the light source can be configured to provide up to 600 mW. The total power consumption of the microscope may not exceed about 1000 mW, 500 mW, 400 mW, 300 mW, 200 mW, 100 mW, 90 mW, 80 mW, 70 mW, 60 mW, 50 mW, 40 mW, 30 mW, 20 mW, 10 mW, 5 mW, or 1 mW.

In some cases, two or more light sources can be provided. A first light source and a second light source can be turned on (e.g., emit light) at the same time. A first light source and a second light source can be alternately pulsed. The first light source can emit light in a first range of wavelengths (e.g., first color) and the second light source can emit light in a second range of wavelengths (e.g., second color). The light from the first light source and/or from the second light source can be delivered to the sample.

In use, the probe can be inserted into an object for imaging by the stabilization device. The object can be an organ and/or tissue of a living organism. In some cases, the living organism can be anesthetized while the probe is inserted into the organ and/or tissue. A hole can be drilled into a surface to permit insertion of the probe. For example, a hole can be drilled in a skull for insertion of the probe into brain tissue. The probe can be attached to the cuff. The probe can be fitted in the cuff. A surface of the cuff can be coated with an adhesive.

The cuff can be fitted in the clamp. Adhesive on the cuff can be prevented from contacting the clamp by one or more lips and/or chamfered edges provided on the cuff that can be configured to provide a physical barrier between a surface of the cuff coated in adhesive and a surface of the cuff that is in contact with the clamp when the cuff is fitted in the clamp. The screw can be tightened to secure the cuff in the clamp. The clamp can be attached to the stereotaxic manipulator rod. In some cases, at least a fraction of the components of the microscope can be mounted on the stabilization device. At least a fraction of the components of the microscope can be mounted on the clamp.

Using the stereotaxic manipulator rod to move the stabilization device, a user can insert the probe into the object for imaging. When the probe is inserted for imaging the cuff can be bonded to an outer surface of the object. In some cases, the outer surface can be a bone surface. The outer surface can be an outer surface of a skull. The probe can be inserted into the object while providing continuous imaging of one or more features in the imaging plane of the probe. A user can monitor the images provided by the probe during insertion and stop the insertion at a desired insertion depth and/or location when a sample of interest is detected. A sample of interest can comprise one or more cells (e.g., cancer cells, neurons, and/or cells that are emitting a predetermined type of fluorescence). Once the sample of interest is detected in the field of view of the probe the angle of the probe can be further adjusted to a desired angle of viewing of the sample. The angle can be adjusted without moving parts. The angle can be adjusted by augmenting a voltage bias across a liquid crystal provided on the probe.

In some cases, once a secure bond between the cuff and a surface of the object has formed, the clamp can be removed from the cuff. The clamp can be removed from the cuff by loosening the screw that connects the cuff to the clamp. The probe can be undisturbed while the clamp and cuff are disconnected. The clamp and cuff can be disconnected with by applying a low torque to the screw such that the probe does not move, shift, rotate, or jostle when the clamp and cuff are disconnected. In some instances, the torque is equal to or less than about 0.0005 Nm, 0.001 Nm, 0.002 Nm, 0.003 Nm, 0.004 Nm, 0.005 Nm, 0.006 Nm, 0.007 Nm, 0.008 Nm, 0.009 Nm, 0.01 Nm, 0.012 Nm, 0.014 Nm, 0.016 Nm, 0.018 Nm, or 0.02 Nm. In some case, when the probe is inserted into live tissue preventing the cuff from moving while disconnecting the clamp and cuff can prevent damage to the tissue.

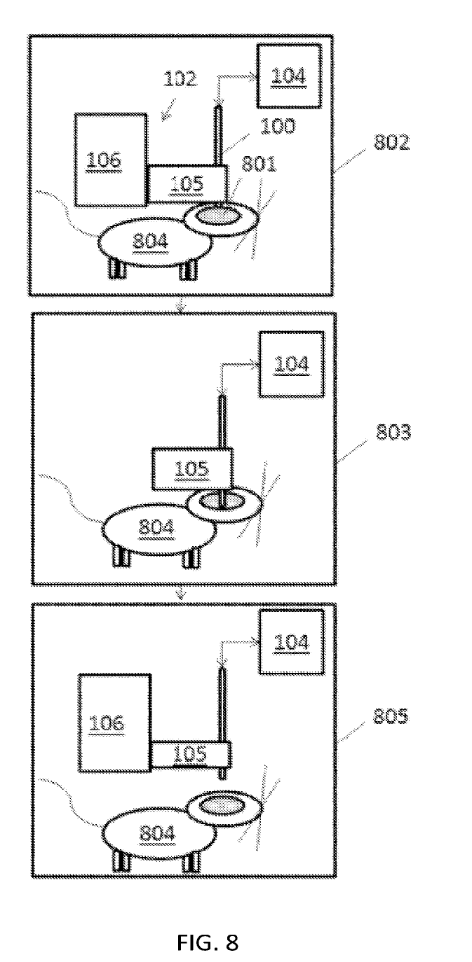

FIG. 8 shows a process of using a stabilization device 102 to insert a probe 100 into an object 804. The steps shown in FIG. 8 can be performed in a different order from the order shown and described herein. In some cases, one or more steps can be added or removed. The object can be an organ 801 of a living being 804. The living being can be a mammal. The living being can be a rodent. In some cases, the living being can be a mouse. The organ of the living being can be a brain. In a first step 802, the stabilization device comprising the clamp and the stereotaxic manipulator rod (not shown) can securely hold the cuff and/or the probe in line with an optical access of the microscope during insertion into the object. The stereotaxic manipulator rod can control placement and/or positioning of the probe during insertion. Maintaining the probe in line with the optical access of the microscope can permit imaging with the microscope while the probe is inserted into the object. The stabilization device can hold the cuff while the cuff is being adhered to a surface that is an outside surface of the object or a surface near the object. The surface can be a surface of the object. In some cases, the cuff can be adhered to a surface that coats, covers, contacts, or resides next to the object.

A user can observe an image captured by the optical probe as the probe is inserted into the object in real time. When the user observes the sample of interest in the field of view of the optical probe, the user can stop insertion of the probe. Once the sample of interest is observed at a given probe insertion distance the user can disconnect the cuff from the clamp as show in step 803. The user can collect images in an imaging session while the probe is inserted into the object a given distance. The imaging session may comprise the collection of one or more images over a continuous period of time. The imaging session may have a duration of less than 1 minute, less than 5 minutes, less than 10 minutes, less than 30 minutes, less than 1 hour, less than 2 hours, less than 5 hours, less than 10 hours, less than 1 day, less than 2 days, less than 5 days, less than 10 days, or less than 30 days. The imaging session may have a duration in a range defined by any two of the preceding values. An imaging session may be defined by a length of time during which a microscope is collecting data. An imaging session may begin when a microscope starts generating images and may end when the microscope stops generating images. An imaging session may begin when the microscope is turned on and may end when the microscope is turned off. A probe position may or may not be altered during an imaging session. The imaging session may be defined by a length of time while a probe is inserted into the object. The imaging session may start when insertion of the probe into the object begins, and may end when the probe is removed from the object. The position of the probe may be altered during the imaging session. A single imaging session or multiple imaging sessions may occur during a longitudinal study. The cuff can remain attached to the surface after the cuff is removed from the clamp. The cuff can be removed from the clamp without jostling or shifting the position of the probe in the object.

When the user is finished collecting imaging, the user can reattach the cuff to the clamp as shown in step 805. The stabilization device comprising the stereotaxic manipulator rod and the clamp can perform a controlled extraction of the imaging probe to remove the probe from the object.

In some instances, the user may desire to go back and image the same area (e.g., the same sample of interest) and/or a same field of view previously imaged. For example, hours, days, week, months, and/or years after removal of the probe, the user may desire to go back and image the same sample of interest previously imaged. As previously described herein, a computer system can comprise a memory storage device. A map of an area containing a subject to be imaged by the device can be stored on the memory storage device. The map can comprise spatial and or relational data pertaining to a tissue, organ, and or bone structure of an animal. In some instances, computer system may store information regarding a placement of the probe and reinsert the probe into the same region for imaging of the same sample of interest (e.g., based on the map).

In some instances, the sample of interest may be recognized by a feature (e.g., landmark feature) or structure inside of the object that is of interest to a user of the probe. In some instances, the recognizable feature or structure may comprise a specific structure such as a valve, node, cell mass, membrane or other structure inherent to the tissue type. In some instances, the recognizable feature or structure may comprise a structure that is associated with an abnormality such as a tumor, cell mass, cyst, ulcer, polyp, fluid mass, or any other tissue abnormality. In some instances, the recognizable feature may comprise a collection of one or more cells (e.g., neurons) or a pattern of cells. For example, a unique pattern of arteries or other features may be formed. The feature or structure may be recognized (e.g., via software, image recognition, image processing, etc) and verified to be the same sample of interest previously imaged. The recognition and verification may happen in real time. In some instances, the recognition and verification may happen during the insertion of the probe. The recognition and verification may happen in real-time. One or more processors may analyze images of one or more interior features of an object. The analysis may occur during the insertion of the probe into the object. The one or more processors may calculate a relative position of the probe compared to one or more landmarks. The one or more processors may send an indication of the relative position, which may be displayed to a user. If a user is targeting having the probe reinserted into a position that it had traversed before, the one or more processors may send the indication whether the probe is on the same path, which may be displayed to a user. In some instances, suggestions may be made as to how to adjust a positioning of the probe to get it back on-track. In some instances, the display may show a user a live-feed of the image captured using the probe. In some instances, visual landmarks or features may be highlighted or otherwise emphasized.

In some instances, only when the desired sample of interest is recognized and verified may the probe be fully inserted to a targeted position. In some instances, while reinserting the probe, if the desired sample of interest is not recognized at an expected position, the probe may be removed in order to minimize potential damage.

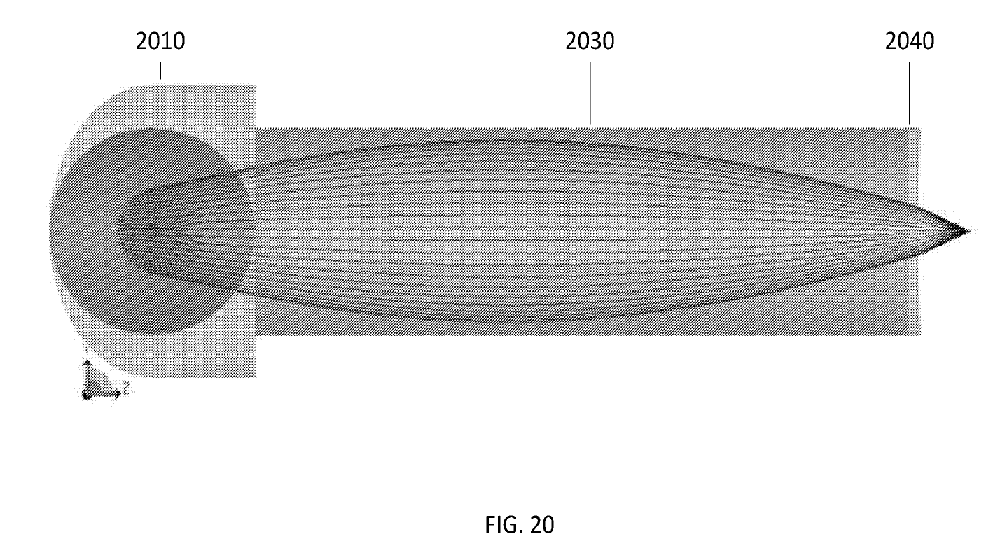

FIG. 20 shows ray traces in a view of one embodiment of a cylindrical prism probe. The cylindrical prism probe may be shaped or optimized for a constrained toroidal object field. The prism probe may comprise a prism 2010, GRIN lens 2030, and corrective optical element 2040. The corrective optical element may have a shape, or best form for correcting aberrations caused by a cylindrical glass/tissue interface. In some instances, the corrective optical element may be concave along one side. In some instances, the corrective optical element may have the form of a stretched bowl. Optionally, a corrective element having a form of a stretched bowl may be appropriate or suited when the corrective element has no net optical power. In some instances, the corrective optical element may have a form in which it is convex along both sides, or both axes. Optionally, a corrective element having a form in which it is convex along both sides may be appropriate or suited when the corrective element has a net optical power. In other instances, the corrective optical element may have a depth equal to or less than about 1000 μm, 800 μm, 600 μm, 500 μm, 400 μm, 300 μm, 250 μm, 200 μm, 150 μm, 100 μm, 75 μm, 50 μm, 40 μm, 30 μm, 20 μm, or 10 μm. The corrective optical element may optionally have a depth such that it may be manufactured on a wafer scale by lithography, e.g. grayscale lithography. In some embodiments, the corrective optical element may have the form of a stretched bowl with a depth of approximately 50 μm. The corrective optical element may be at an opposing end of the GRIN lens relative to the prism. The corrective optical element may directly contact the GRIN lens. The corrective optical element may be separable from the GRIN lens or may be permanently affixed or integral to the GRIN lens. The corrective optical element may be glued to the GRIN lens. The corrective optical element may be forcefully held against the GRIN lens. For instance, the corrective optical element may be forcefully held against the GRIN lens using a reinforcing sleeve. The ray trace shows the correction of imaging artifacts afforded by the addition of the corrective optical element.

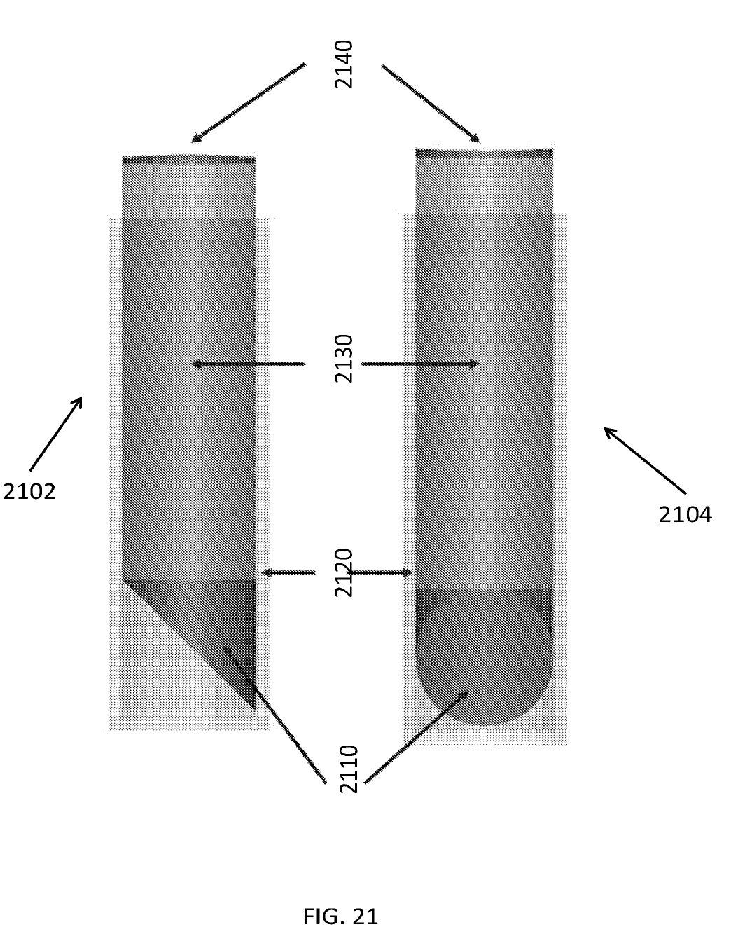

FIG. 21 illustrates one embodiment of the cylindrical prism probe set to a maximum depth within an implantable cannula. The maximum depth corresponds to the position at which the prism probe no longer has room to travel within the cannula. Embodiment 2102 presents a side view. Embodiment 2104 presents a front view. The prism probe may comprise a prism 2110, cannula 2120, GRIN lens 2130, and/or corrective optical element 2140. The cannula may be part of the prism probe or may be separate from the prism probe. The corrective optical element may have the form of a stretched bowl. In some embodiments, the corrective optical element may have the form of a stretched bowl with a depth of approximately 50 μm. The corrective optical element may have any the characteristics described elsewhere herein.

When at a maximum depth, a distal end of the GRIN lens may contact an interior bottom surface of the cannula. The distal end of the GRIN lens, or a prism at the distal end of the GRIN lens may be flat or angled. When an end of the GRIN lens or the prism is angled, there may be an unoccupied space within the cannula between the angled surface and the interior bottom surface of the cannula. The sides of the GRIN lens may fit snugly within the implantable cannula. As previously described, the sides of the GRIN lens may or may not directly contact the interior surfaces of the implantable cannula. The corrective optical element may be provided at a proximal end of the GRIN lens. The corrective optical element may or may not be within the cannula. In some embodiments, the length of the GRIN lens may be greater than the length of the cannula. In some embodiments, the length of the GRIN lens plus the length of the prism may be greater than the length of the cannula. The length of the GRIN lens may be sufficiently great such that when the prism probe is fully inserted into the cannula, the corrective optical element and/or the GRIN lens are not within the cannula.

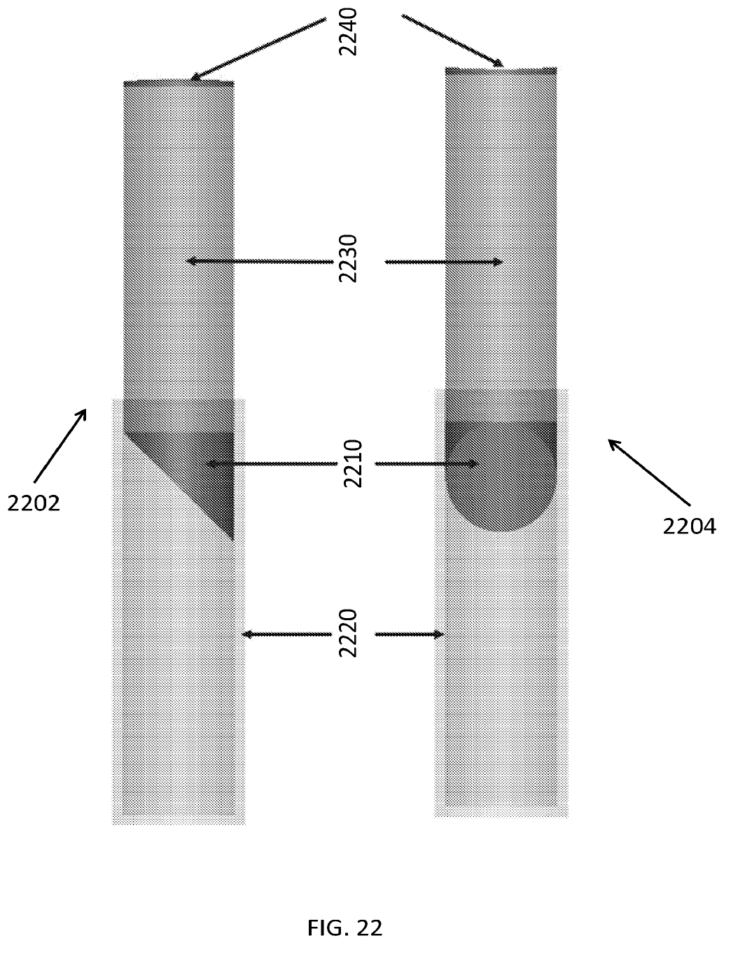

FIG. 22 illustrate one embodiment of a cylindrical prism probe set to a reduced depth within an implantable camera. In some embodiments, the reduced depth may be a minimum depth within an implantable cannula. The minimum depth corresponds to the position at which further withdrawal of the prism probe would remove the prism probe from the cannula entirely. Embodiment 2202 presents a side view. Embodiment 2204 presents a front view. The prism probe may comprise a prism 2210, cannula 2220, GRIN lens 2230, and/or corrective optical element 2240. The cannula may be part of the prism probe or may be separate from the prism probe. The corrective optical element may have any the characteristics described elsewhere herein.

When at a reduced depth, a distal end of the GRIN lens and/or prism may not contact an interior bottom surface of the cannula. The distance between a distal end of an angled surface or a proximal end of the angled surface and the interior bottom surface of the cannula may be substantially the length of the cannula. There may be an unoccupied space within the cannula between an angled surface and the interior bottom surface of the cannula. The sides of the GRIN lens may fit snugly within the implantable cannula. As previously described, the sides of the GRIN lens may or may not directly contact the interior surfaces of the implantable cannula. When at the minimum depth, the sides of the GRIN lens may be outside of the cannula or mostly outside of the cannula except for the distal end. An angled surface may be within the cannula or may be substantially outside of the cannula.

### EXAMPLE

### Design and Testing of a Microendoscopic “Prism Probe”

Design and testing of the prism probes of the present disclosure has been undertaken to address the need for implantable endoscopes whose viewing depth and viewing direction can be changed at any time following implantation. In some embodiments, the prism probe is designed to fit snugly within an implantable, cylindrical glass cannula so that depth and direction may be adjusted. In some instances, the snug fit is characterized by contact on all sides between the prism probe and the cannula. In some cases, the snug fit arises due to a friction fit. In some cases, the snug fit is characterized by the absence of a gap between the prism probe and the cannula. In some cases, the snug fit is characterized by a gap no more than 0.001 mm, 0.002 mm, 0.005 mm, 0.01 mm, 0.02 mm, 0.05 mm, or 0.1 mm between the prism probe and the cannula. In some embodiments, the prism probe and a miniature microscope to which it is optically coupled or attached may be raised or lowered (or rotated) as a unit relative to the position of the cannula. The prism probe itself may comprise a cylindrical prism, a GRIN lens, and a corrective optical element that compensates for optical aberration. In some embodiments, the prism is fabricated by grinding or etching a 45° flat surface at one end of the GRIN lens. In some embodiments, a corrective optical element is fitted to the end of the GRIN lens opposite that which is attached to a prism or that has been ground or etched to a 45° flat surface.

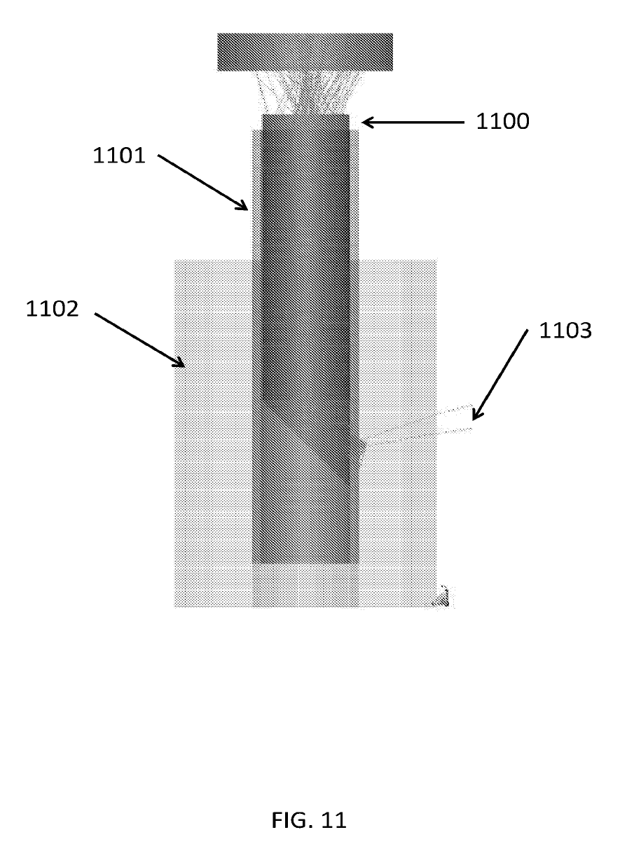

FIG. 11 illustrates one embodiment of a prism probe 1100 that comprises a 1 mm diameter cylindrical 45 degree prism and a GRIN lens that fits inside an implantable glass cannula 1101 to provide adjustable viewing depth and viewing direction. For testing purposes, the glass cannula was sometimes immersed in a water bath 1102. Light rays entering the GRIN lens are refracted and directed to the prism. Upon interaction with the prism, the light rays are directed through the cannula and into the material in which the cannula is implanted. In some instances, the light rays are directed at an angle upon interaction with the prism as shown by ray 1103.

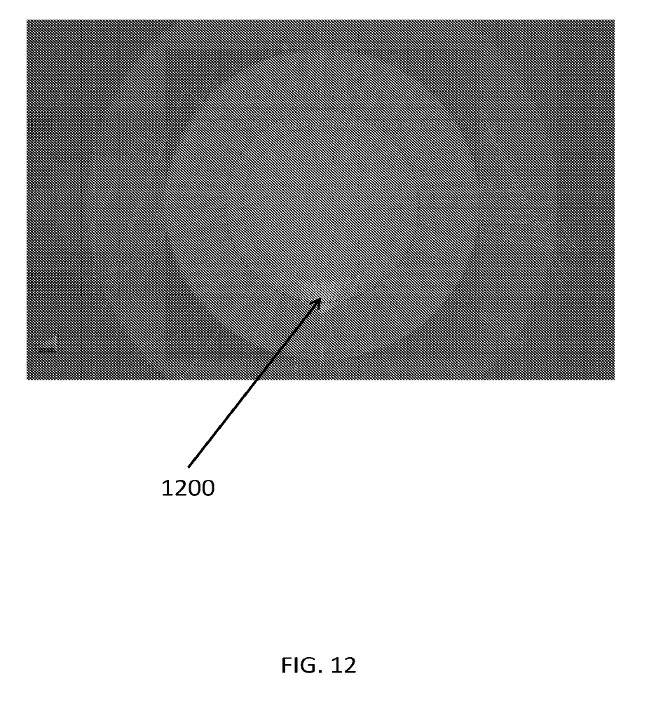

FIG. 12 illustrates the refraction of light rays at the interface between the implanted cannula wall and tissue (1200) that gives rise to image blur and distortion. As indicated by the dashed circle, the image blur and distortion arises as a result of the refraction of light between the implantable probe and the cannula. The refraction of light arises from a difference in indices of refraction of the probe and the cannula.

In order to correct for the image blur and distortion arising from the cylindrical cannula-tissue interface, a toroidal refractive element (i.e., a corrective optical element) was introduced to the prism probe design. Two locations for the corrective element were investigated: (i) at the midpoint of the GRIN lens, and (ii) at the end of the GRIN lens opposite the 45° prism or face. Subsequent studies have focused on designs in which the corrective optical element is placed at the end of the GRIN lens, as this approach provides for simpler coupling of the corrective element with the GRIN lens, e.g., in some embodiments, the corrective element may simply be glued to the end of the GRIN lens using an optical adhesive.

For designs in which the corrective optical element was placed at the end of the GRIN lens, two design optimization approaches were investigated for improving image resolution. In the first, a design goal of achieving a flat object field was imposed, and the shape (or form) of the toroidal optical corrective element was optimized accordingly. In the second approach, the constraint on the object field was relaxed, e.g., the object field was allowed to be toroidal, and the best combination of object field shape and corrective optical element form was sought. For optimization purposes, the prism probe design was analyzed independently of an attached miniature microscope using the following set of design optimization parameters: object field position (x, y) ranging from (0, 0) to (0.212 mm, 0.212 mm); image distance fixed at 0.25 mm from the end of the GRIN lens; root-mean-square (RMS) spot radius was minimized at the intermediate focal plane; while the length of the GRIN lens, the radii of the toroidal corrective element, and optionally the object field radii were allowed to vary.

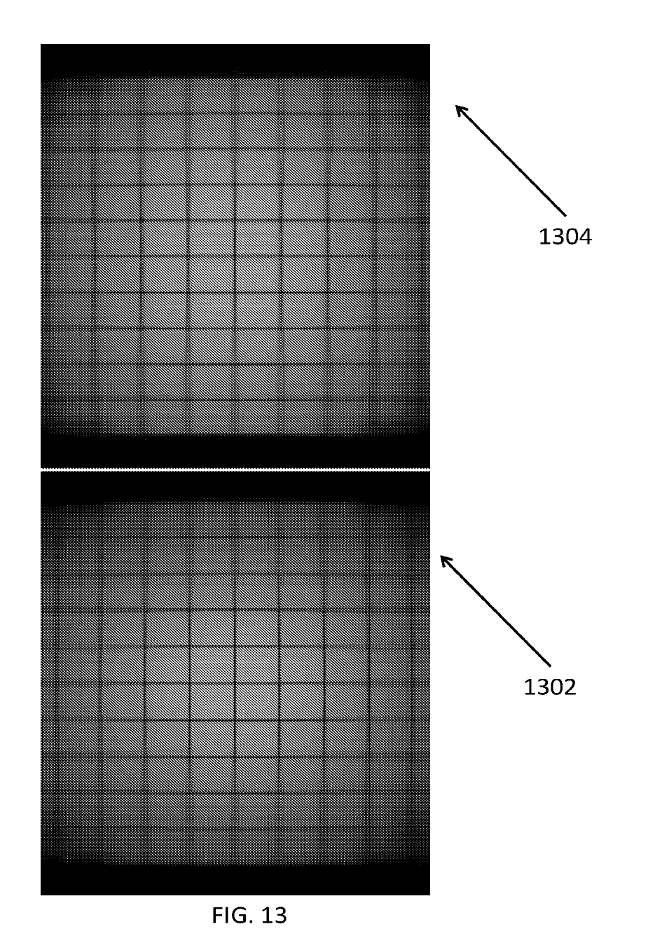

FIG. 13 shows a comparison of the image distortion observed for an uncorrected 1 mm square prism probe to that observed for an uncorrected 1 mm cylindrical prism probe. Embodiment 1302 shows an image of a 100 micron pitch grid captured at the intermediate focal plane using an uncorrected 1 mm square prism probe having a flat object field. The images were formed using a ray tracing procedure. Embodiment 1304 shows an image of a 100 micron pitch grid captured at the intermediate focal plane using an uncorrected 1 mm cylindrical prism probe having a flat object field. Although the cylindrical probe had worse resolution near field center due to the astigmatism of the cylindrical cannula-tissue interface, the off-axis performance was not significantly worse.

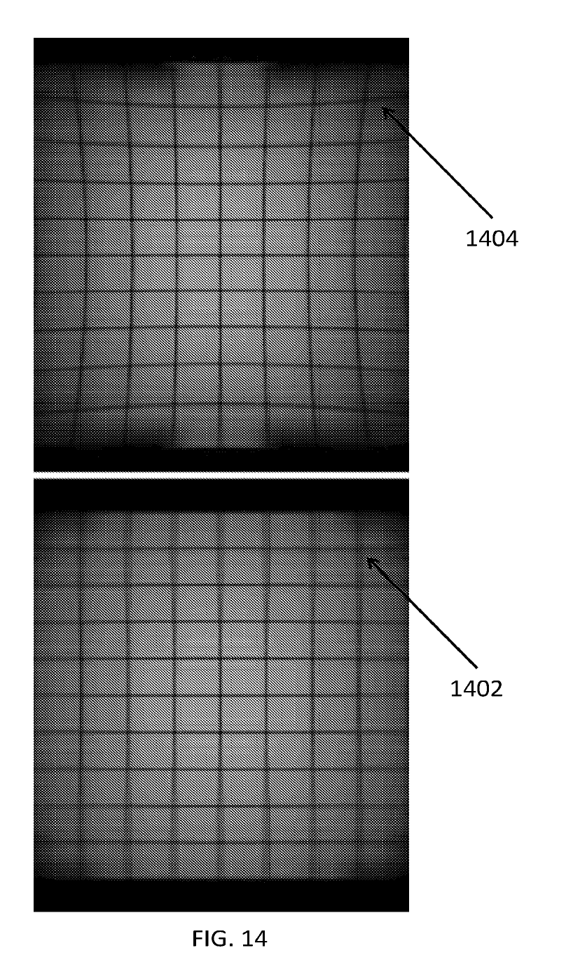

FIG. 14 shows a comparison of the image distortion observed for images captured using cylindrical prism probes having corrective optical elements attached. Embodiment 1402 shows an image of a 100 micron pitch grid captured using a 1 mm cylindrical prism probe comprising a corrective optical element optimized for a flat object field. A resolution of 1.4 μm was achieved at field center, and a resolution of 13 μm was achieved at a radius of 0.4 mm. The distortion in the image is 0.6%. The “surface sag” of the corrective optical element was 20 μm. Embodiment 1404 shows an image of a 100 micron pitch grid captured using a 1 mm cylindrical prism probe comprising a corrective optical element optimized for a toroidal object field. A resolution of 0.9 μm was achieved at field center, while a resolution of 6.4 μm was achieved at a radius of 0.4 mm. The distortion in the field is 6%. The surface sag of the corrective optical element was 40 μm, while that of the object field was 50 μm (object field radii=0.75 mm and 1.0 mm).

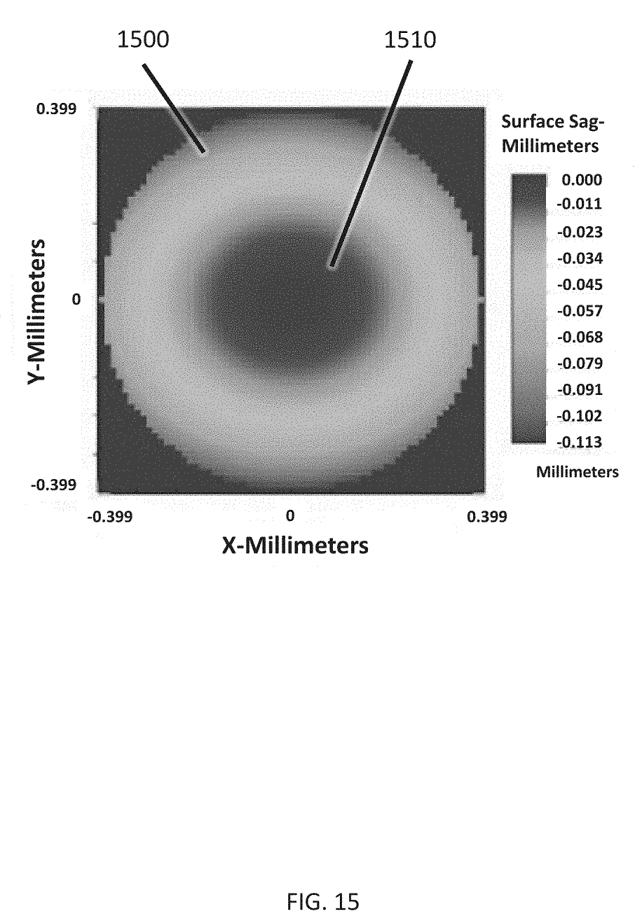

FIG. 15 shows a plot of toroidal object field surface sag as a function of field position. The object field has the form of a stretched bowl with a depth of approximately 100 μm. The stretched bowl has an outer region 1500 having a surface sag of approximately 100 μm and an inner region 1510 having a surface sage of approximately 0 μm. The surface sag varies from 0 μm to approximately 100 μm in the areas between the outer region and the inner region.

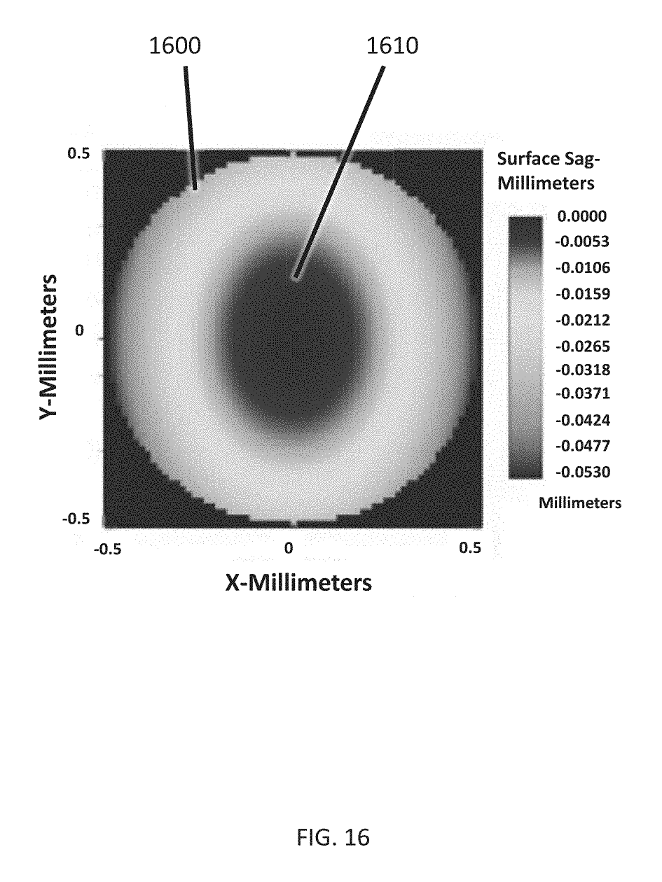

FIG. 16 shows a plot of corrective optical element surface sag as a function of position for the corrective optical element used to achieve the unconstrained toroidal object field shown in FIG. 15. The corrective optical element also has the form of a stretched bowl with a depth of approximately 50 μm. The corrective element thus has a net optical power but has a depth that is incompatible with wafer-scale manufacturing. The stretched bowl has an outer region 1600 having a surface sag of approximately 50 μm and an inner region 1610 having a surface sage of approximately 0 μm. The surface sag varies from 0 μm to approximately 50 μm in the areas between the outer region and the inner region.

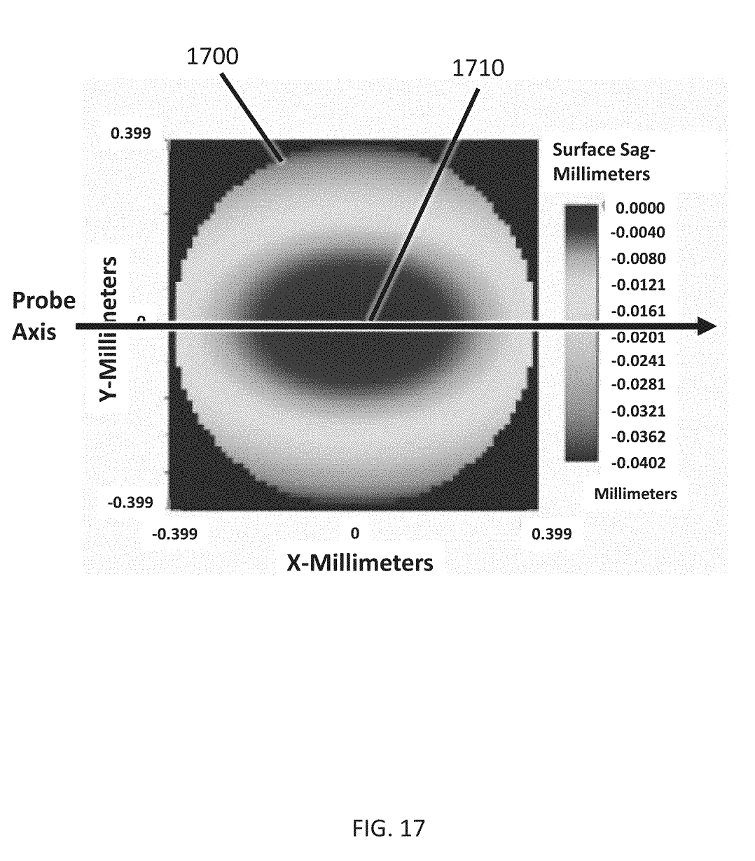

FIG. 17 shows a plot of object field surface sag as a function of field position when the radii of curvature of the toroidal object field were constrained to values of −2 mm and −5 mm respectively. The object field is nearly cylindrical and has the form of a stretched bowl with a depth of approximately 40 μm. The prism probe axis is indicated by the arrow. The stretched bowl has an outer region 1700 having a surface sag of approximately 40 μm and an inner region 1710 having a surface sage of approximately 0 μm. The surface sag varies from 0 μm to approximately 40 μm in the areas between the outer region and the inner region.

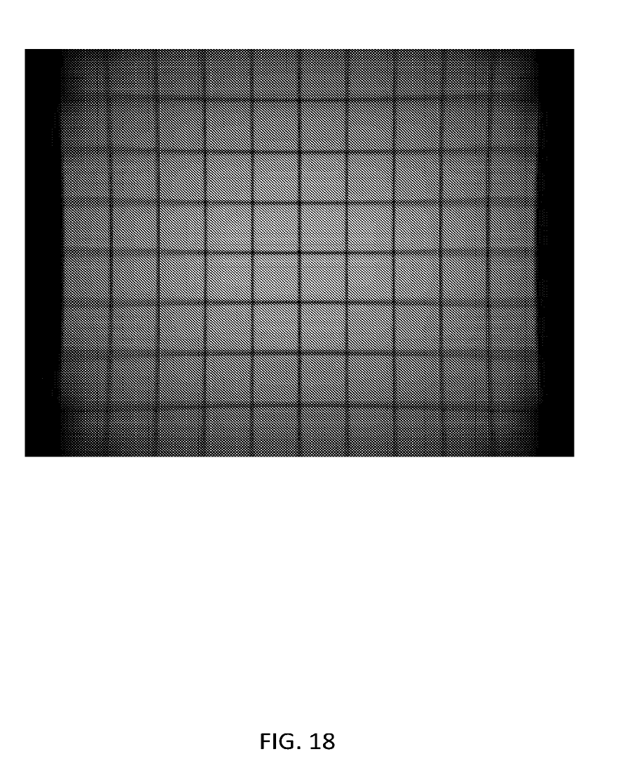

FIG. 18 shows an example of image distortion for an image of a 100 micron pitch grid captured using a 1 mm cylindrical prism probe comprising a cylindrical corrective optical element designed to achieve a constrained toroidal object field. A resolution of 1.4 μm was achieved at field center, while a resolution of 10 μm was achieved at field edge.

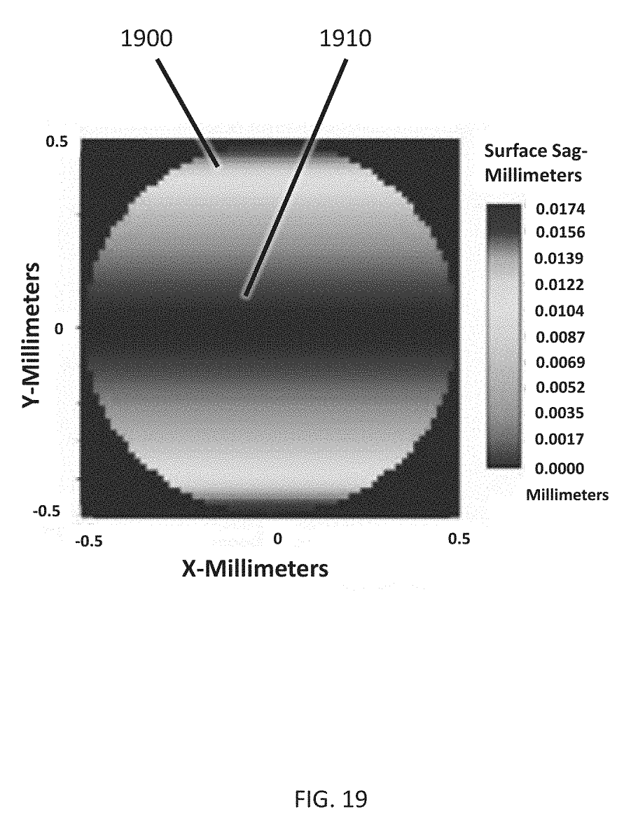

FIG. 19 shows a plot of surface sag as a function of position for the cylindrical corrective optical element used to obtain the image of FIG. 18. The corrective element has the form of a saddle with a depth of approximately 18 μm. The saddle has an outer region 1900 having a surface sag of approximately 18 μm and an inner region 1910 having a surface sage of approximately 0 μm. The surface sag varies from 0 μm to approximately 18 μm in the areas between the outer region and the inner region.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-35. (canceled)

36. A method of implanting an optical probe into an object for imaging one or more interior features of the object, the method comprising:
supporting an optical probe with a stabilization device, wherein the optical probe is in optical communication with a microscope;
inserting at least a portion of the optical probe into the object, while the optical probe is supported by the stabilization device;
continuously imaging, with aid of the microscope, one or more interior features of the object while inserting the portion of the probe into the object; and
displaying the one or more interior features of the object while using the microscope and inserting the probe into the object.

37. The method of claim 36, wherein the stabilization device is connected to a stereotaxic manipulator rod configured to allow movement of the optical probe with respect to at least three axes or allow translation or rotation of the optical probe.

38. The method of claim 36, wherein the stabilization device comprises a clamp connected to a cuff, wherein the cuff is supporting the optical probe, and the method further comprises removing the clamp from the cuff while maintaining the location of the probe.

39. The method of claim 36, wherein a field of view of the optical probe is changed while inserting the probe into the object.

40. The method of claim 36, wherein the one or more interior features of the object are displayed on a display terminal in real time.

41. A device configured to implant an optical probe into an object for imaging, the device comprising:
a cuff that supports an optical probe that is in optical communication with a microscope, the cuff (1) comprising a surface that connects to an outer surface of the object with an adhesive and (2) configured to prevent adhesive from leaking out of a contact area between the (a) surface of the cuff and (b) the outer surface of the object; and
a clamp that removably connects to the cuff and is configured to connect to a stereotaxic manipulator rod configured to control the device when the optical probe is inserted into the object.

42. The device of claim 41, wherein the optical probe is configured to collect one or more images while the optical probe is being inserted into the object.

43. The device of claim 41, wherein the device is configured to locate a feature of interest while the optical probe is being inserted into the object.

44. The device of claim 41, wherein the stereotaxic manipulator rod is configured to allow movement of the optical probe with respect to at least three axes or allow translation or rotation of the optical probe.

45. The device of claim 41, wherein the optical probe comprises a relay lens.

46. The device of claim 45, wherein the relay lens is a gradient index lens having a surface with a 45 degree angle.

47. The device of claim 45, wherein the relay lens is located at a distal end of the optical probe.

48. The device of claim 45, wherein the optical probe comprises a corrective optical element for correcting optical aberration.

49. The device of claim 48, wherein the corrective optical element comprises a refractive or diffractive optical element.

50. The device of claim 48, wherein the corrective optical element is designed to provide a toroidal object field.

51. The device of claim 41, wherein the optical probe comprises an optical element configured to alter a viewing angle of the optical probe.

52. A method of accessing an interior of an object for imaging with an optical probe, the method comprising:
supporting an optical probe relative to the object, wherein the optical probe is in optical communication with a microscope;
imaging, with aid of the microscope, at a plurality of different depths in the object within a single imaging session; and
imaging, with aid of the microscope, at a plurality of different fields of view within the single imaging session, wherein the plurality of different fields of view are determined by rotating the optical probe about a longitudinal axis; and
wherein the optical probe comprises: (1) an aberration correction element at a proximal end of the optical probe, and (2) an angled surface at a distal end of the optical probe that forms an angle between 30-60 degrees relative to a length of the optical probe, such that a field of view imaged by the microscope is not co-linear to the length of the optical probe, and wherein the angled surface covers an entirety of the distal end of the optical probe.

53. The method of claim 52, wherein of the optical probe comprises an optical element having the angled surface at a distal end of a lens.

54. The method of claim 53, wherein the lens is a GRIN lens, and wherein the angled surface forms an angle of 45° with respect to the optical axis of the GRIN lens.

55. The method of claim 52, further comprising correcting monochromatic or poly-chromatic optical aberrations with aid of refractive or diffractive optical elements of the optical probe, wherein the microscope achieves an image resolution of at least 2 micrometers (μm) across the field of view.
